(19) United States
(12) Patent Application Publication
(10) Pub. No.: US 2022/0242863 A1
(43) Pub. Date: Aug. 4, 2022

(54) COMPOUNDS FOR TREATMENT OF EYE DISORDERS

(71) Applicant: SINOPSEE THERAPEUTICS, Singapore (SG)

(72) Inventors: Srinivasaraghavan KANNAN, Singapore (SG); Hong Hwa LIM, Singapore (SG); Chandra Shekhar VERMA, Singapore (SG); Uttam SURANA, Singapore (SG)

(21) Appl. No.: 17/596,976

(22) PCT Filed: Jun. 25, 2020
(86) PCT No.: PCT/SG2020/050363

§ 371 Date: Dec. 22, 2021

(30) Foreign Application Priority Data
Jun. 25, 2019 (SG) 10201905890P

(51) Int. Cl. C07D 471/04; C07D 487/04; A61P 27/00

(52) U.S. Cl. CPC C07D 471/04; A61P 27/00; C07D 487/04

(57) ABSTRACT

Compounds of formula I as defined herein, or pharmaceutically acceptable salts, solvates or derivatives thereof, are potent inhibitors of angiogenesis and accordingly are of use in the treatment and prevention of various angiogenesis-related disorders such as cancer.

### FIELD OF INVENTION

The present invention relates to the field of pharmaceuticals, and in particular to compounds which are inhibitors of angiogenesis. Compounds of the invention may be useful in the treatment of: angiogenesis and angiogenesis-related disorders, such as eye disorders (e.g. macular degeneration and diabetic retinopathy).

### BACKGROUND

The listing or discussion of a prior-published document in this specification should not necessarily be taken as an acknowledgement that the document is part of the state of the art or is common general knowledge.

Macular degeneration “MD” is a disease that affects a layer of cells in the eye known as the retinal pigment epithelium, which lies underneath the retina. The retinal pigment epithelium (RPE) acts as a wall or barrier and is responsible for passing oxygen, sugar and other essentials up to the retina and moving waste products down to the blood vessels underneath (these vessels are called the choroid). The RPE also acts as a barrier between the choroid and the retina. When RPE cells die, the retinal cells above them also die, leading to patches of ‘missing’ retina. This is commonly called geographic atrophy or “dry” MD, which is a slow form of the disease that causes a gradual loss of vision. “Wet” macular degeneration occurs when the RPE cells fail to stop choroidal blood vessels from growing under the retina. This growth is called choroidal neovascularisation or CNV. The rapidly growing vessels are fragile with leaky walls and they ooze fluid and blood under the retina. This leads to scarring and severe loss of central vision, which if left untreated, becomes permanent. In the context of the present invention it will be appreciated that the term “macular degeneration” particularly refers to “wet” MD also known as neovascular or exudative AMD, but also includes “dry” MD.

Diabetic retinopathy is a microvascular complication of diabetes which can occur in the eye. There are multiple categories and classifications of diabetic retinopathy, for example the earlier stage of nonproliferative diabetic retinopathy (NPDR) and the advanced stage of proliferative diabetic retinopathy (PDR) associated with abnormal blood vessel growth. Diabetic macular edema (DME) is also included within its scope. DME is a manifestation of diabetic retinopathy that occurs across all severity levels of both NPDR and PDR and represents the most common cause of vision loss in patients. DME arises from diabetes-induced breakdown of the blood-retinal barrier (BRB), with consequent vascular leakage of fluid and circulating proteins into the neural retina. The extravasation of fluid into the neural retina leads to abnormal retinal thickening and often cystoid edema of the macula.

In wet AMD and diabetic retinopathy, VEGFα is believed to play a significant role in the formation of blood vessels that grow abnormally and leak beneath the macula. The constant exposure of endothelial cells to pro-angiogenic factors, such as VEGFα, result in the formation of immature, semi-differentiated and fragile blood vessels which have a tendency to leak and bleed. The three main receptor tyrosine kinases (RTKs) responsible for abnormal blood vessel growth in the context of MD are PDGFRα, PDGFRβ and VEGFR2. These receptor tyrosine kinases are high affinity cell surface receptors for polypeptide growth factors such as VEGFα. Accordingly, it is believed that the compounds which can distinguish between “diseased” and normal cells may exhibit a wider therapeutic window than compounds or agents that do not.

### SUMMARY OF THE INVENTION

Without wanting to be bound by theory, the present invention is based on the surprising finding that a compound of formula I as defined herein displays high selectivity towards the receptor tyrosine kinases (RTKs) PDGFRα, PDGFRβ and VEGFR2. As such, compounds of formula I are believed to be particularly well suited for therapeutic application to patients with macular degeneration as they may be able to inhibit proliferation of only “diseased” cells; i.e. with high density of receptor tyrosine kinases. It is believed that the compounds of the present invention may be effective in blocking the sprouting of abnormal blood vessel formation, and accordingly be advantageous for treating MD and/or diabetic retinopathy.

The present invention therefore provides the following numbered clauses.

1. A compound of formula I:

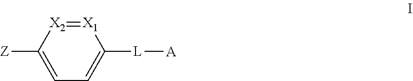

wherein:
X₁ and X₂ each independently represent N or CRₐ
R₈ independently represents H, NH₂, halo, C₁₋₅alkyl, C₁₋₅alkoxy, C₂₋₅ alkenyl and C₂₋₅alkynyl (which latter four groups are unsubstituted or substituted by one or more halo substituents);
A is selected from the group consisting of:

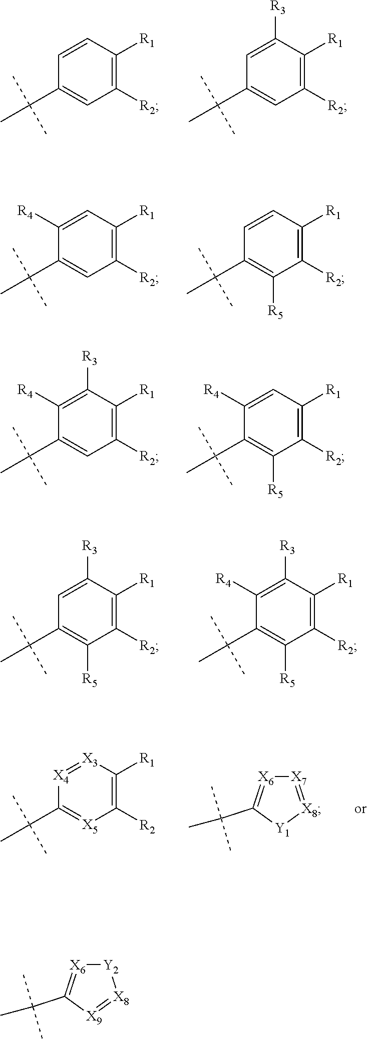

where:
the dotted line represents the point of attachment to the rest of the molecule;
each R₁ to R₅ is independently selected from halo, C₁₋₅ alkyl, C₁₋₅ alkoxy, C₂₋₅ alkenyl, C₂₋₅ alkynyl, which latter four groups are unsubstituted or substituted by one or more halo substituents;
X₃ represents N, CH or CR₃, where R₃ is as defined above, X₄ represents N, CH or CR₄, where R₄ is as defined above, X₅ represents N, CH or CR₅, where R₅ is as defined above, provided that only one or two of X₃ to X₅ is N;
each X₆ to X₉ independently represents N, CH or CR₆, where each R₆ is independently selected from C₁₋₅ alkyl, C₁₋₅ alkoxy, C₂₋₅ alkenyl, C₂₋₅ alkynyl, which four groups are unsubstituted or substituted by one or more halo substituents;
wherein in any moiety A, one of R₁ to R₆ may be piperazine, methylpiperazine or ethylpiperazine, each of which may be connected to the rest of the moiety A via a carbon or nitrogen atom in the piperazine ring;
Y₁ represent NR_{N}, O or S;
Y₂ represents NR_{N}, NR_{Y} O or S;
R_{N} represents H, C₁₋₅ alkyl, C₂₋₅ alkenyl, C₂₋₅ alkynyl, which latter three groups are unsubstituted or substituted by one or more halo substituents;
R_{Y} represents piperazine, methylpiperazine or ethylpiperazine, each of which is connected to the nitrogen atom in Y₂ via a carbon atom in the piperazine ring;
L is a linking group of the formula:

-M-(CR_{L}R_{M})ₐ—C(O)—NR₇—;

-M-(CR_{L}R_{M})ₐ—NR₇—C(O)—; or

-M-C(O)—(CR_{N}R_{O})—C(O)-M-

where M represents a covalent bond, O or NH;
R_{L} and R_{M} each independently represent H, methyl, ethyl, fluoro or chloro, or R_{L} and R_{M} together with the carbon atom to which they are attached, form a C₃ or C₄ cycloalkyl ring, carbonyl or thiocarbonyl group;
a represents 0 or 1;
R₇ and R_{7′} represent H or an optionally substituted alkyl group;
R_{N} and R_{O} each independently represent H, methyl, ethyl, fluoro or chloro;
Z represents a heterocycle selected from the group consisting of:

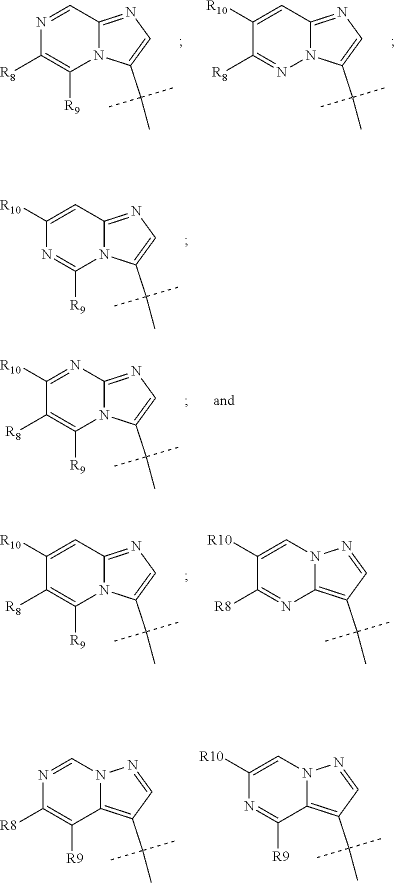

where:
the dotted line represents the point of attachment to the rest of the molecule, and Z is attached to the rest of the molecule via a covalent bond, or via a —O— or —NH— group;
each of R₈ to R₁₀ are independently selected from H, hydroxy, C₁ to C₅ alkyl, C₁ to C₅ alkoxy (which latter two groups are unsubstituted or substituted by one or more halo groups), OC(O)R₁₁, C(O)OR₁₂, C₂ to C₅ alkynyl (which is unsubstituted or substituted by one or more halo groups) or NR₁₃R₁₄, and O—(C₁₋₄ alkyleneyl)-O—C₁₋₄ alkyl,
and one of R₈ to R₁₀ may be a group of the formula:

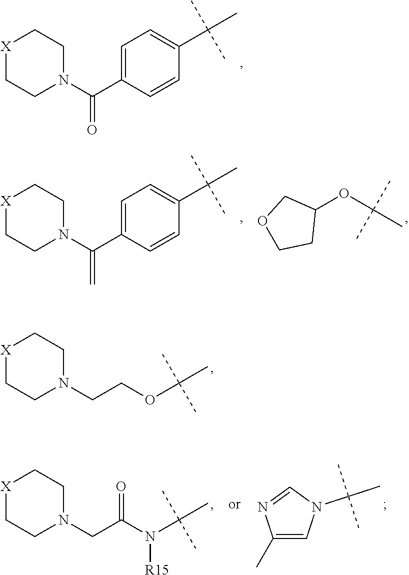

where X represents O, NR_{X},

R_{X} represents H or C₁0.4 alkyl,
R₁₁ and R₁₂ each independently represent, at each occurrence, optionally substituted alkyl;
R₁₃ and R₁₄ each independently represent, at each occurrence, H or optionally substituted alkyl;
R₁₅ represents H or C₁₋₂ alkyl; or
a pharmaceutically acceptable salt, solvate or derivative thereof, provided that when X₁ and X₂ are both CH, L is

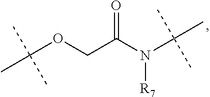

### and A is

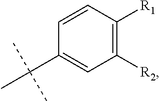

then Z is not an optionally substituted heteroaryl selected from optionally substituted tetrazolyl or optionally substituted imidazopyridinyl.

2. A compound according to Clause 1, or a pharmaceutically acceptable salt, solvate or derivative thereof, wherein R₈ independently represents H, NH₂, F, Cl, or C₁₋₃ alkyl, which C₁₋₃ alkyl group is unsubstituted or substituted by one, two or three fluoro or chloro substituents,

optionally wherein R₈ is H or F.

3. A compound according to Clause 1 or 2, wherein X₁ is selected from N and CH, and X₂ is selected from CH and CF.

4. A compound according to any one of Clauses 1 to 3, or a pharmaceutically acceptable salt, solvate or derivative thereof, wherein:

each R₁ to R₅ independently represents halo, C₁₋₃ alkyl, C₁₋₃ alkoxy, C₂₋₃ alkenyl and C₂₋₃ alkynyl (which four groups are unsubstituted or substituted by one or more halo substituents), optionally wherein each R₁ to R₅ independently represents fluoro, chloro, methyl or ethyl, which methyl and ethyl groups may be unsubstituted or substituted by one, two or three fluoro or chloro groups.

5. A compound according to Clause 1 or 2, or a pharmaceutically acceptable salt, solvate or derivative thereof, wherein:

Y₁ and Y₂ independently represent O, NC₁₋₃ alkyl or NH; and/or
R₆ independently represents C₁₋₃ alkyl, C₁₋₃ alkoxy, C₂₋₃ alkenyl and C₂₋₃ alkynyl (which four groups are unsubstituted or substituted by one or more halo substituents),
optionally wherein Y₁ and Y₂ independently represent O, NMe or NH, and/or
R₆ independently represents fluoro, chloro, methyl or ethyl, which methyl and ethyl groups may be unsubstituted or substituted by one, two or three fluoro or chloro groups.

6. A compound according to Clause 4, or a pharmaceutically acceptable salt, solvate or derivative thereof, wherein each of R₁ to R₅ and R₆ independently represents methyl, trifluoromethyl, fluoro or chloro.

7. A compound according to any one of the preceding Clauses, or a pharmaceutically acceptable salt, solvate or derivative thereof, wherein:

(a) each R₈ to R₁₀ independently represents H, hydroxy, Me, C₁₋₂ alkoxy (which is unsubstituted or substituted by one or more halo groups), OC(O)R₁₁, C(O)OR₁₂, C₂ to C₃ alkynyl (which is substituted by one or more halo groups), O—(C₁₋₂ alkyleneyl)-O—C₁₋₂ alkyl, or NR₁₃R₁₄,

R₁₁ and R₁₂ each independently represent methyl or ethyl,
R₁₃ and R₁₄ each independently represent H, methyl or ethyl; or

(b) one of R₈ to R₁₀ represents a group of the formula

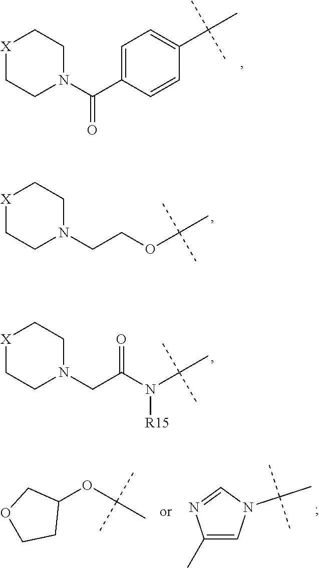

where X represents O, NH, or N—C₁₋₂ alkyl,
R₁₅ represents methyl,
and the remaining two of R₈ to R₁₀ are as defined in part (a).

8. A compound according to any one of the preceding Clauses, or a pharmaceutically acceptable salt, solvate or derivative thereof, wherein Z represents a heterocycle selected from:

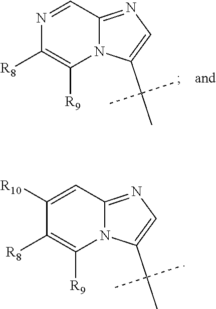

9. A compound according to any one of the preceding Clauses, or a pharmaceutically acceptable salt, solvate or derivative thereof, wherein:

(a) when any of R₈ to R₁₀ is a C₁ to C₅ alkyl group, it is an unsubstituted methyl group; and/or

(b) when any of R₈ to R₁₀ is a C₂ to C₅ alkynyl group, it is a C₂ to C₅ alkynyl group which is substituted by one or more halo groups.

10. A compound according to any one of the preceding Clauses, wherein:

R₉ and R₁₀, when present, are H, and
R₈ is selected from H and

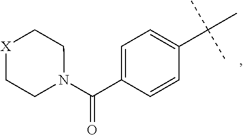

where X is O or NH.

11. A compound according to any one of any one of the preceding Clauses, or a pharmaceutically acceptable salt, solvate or derivative thereof, wherein A is selected from the group consisting of:

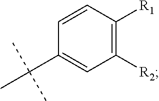

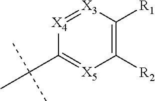

where only one of X₃ to X₅ is N;

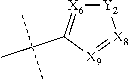

where only one of X₆, X₈ or X₉ is N; or

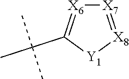

where only one of X₆, X₇ or X₈ is N.

12. A compound according to Clause 11, wherein A is selected from the group consisting of:

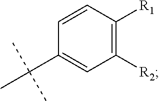

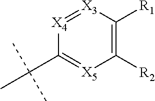

where only one of X₃ to X₅ is N;

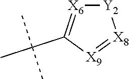

where only one of X₆, X₈ or X₉ is N;

and where when present:
R₁ is selected from Cl and CH,
R₂ is CF₃,
X₃ and X₅ are CH,

### X₄ is N,

### X₆ is N,

X₈ and X₉ are CH,
Y₂ are selected from N—CH₃ and O.

13. A compound according to any one of the preceding Clauses, or a pharmaceutically acceptable salt, solvate or derivative thereof, wherein:

M represents O or NH; and/or
R_{L} and R_{M} each independently represent H, methyl or chloro, or R_{L} and R_{M} together represent thiocarbonyl or cyclopropyl; and/or
a represents 1.

14. A compound according to any one of the preceding Clauses, or a pharmaceutically acceptable salt, solvate or derivative thereof, wherein L represents:

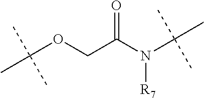

where the dotted lines represent the point of attachment to the rest of the molecule.

15. A compound according to Clause 1 which is selected from:

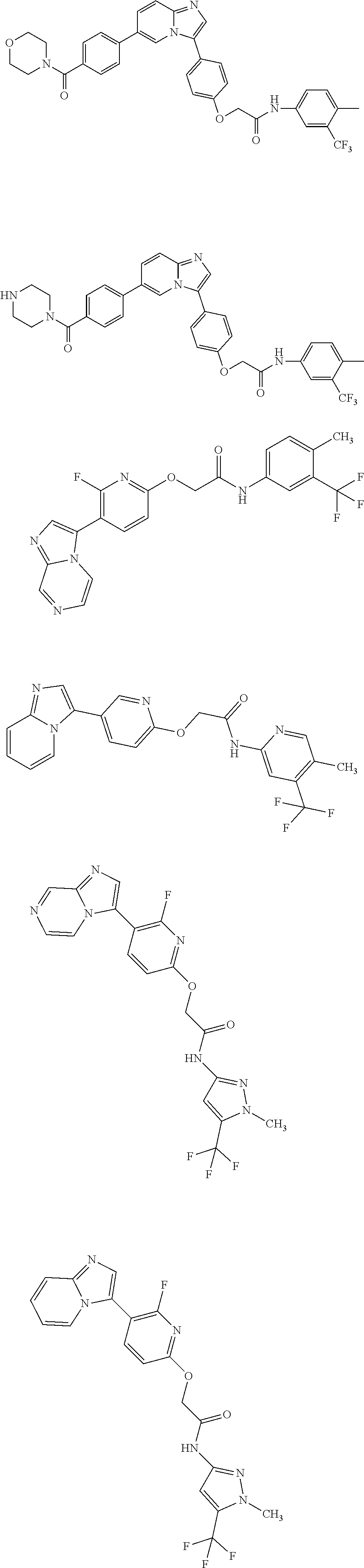 [SMILES: Cc1ccc(NC(=O)COc2ccc(-c3cnc4ccc(-c5ccc(C(=O)N6CCOCC6)cc5)cn34)cc2)cc1C(F)(F)F ; Cc1ccc(NC(=O)COc2ccc(-c3cnc4ccc(-c5ccc(C(=O)N6CCNCC6)cc5)cn34)cc2)cc1C(F)(F)F ; Cc1ccc(NC(=O)COc2ccc(-c3cnc4cnccn34)c(F)n2)cc1C(F)(F)F ; Cc1cnc(NC(=O)COc2ccc(-c3cnc4ccccn34)cn2)cc1C(F)(F)F ; Cn1nc(NC(=O)COc2ccc(-c3cnc4cnccn34)c(F)n2)cc1C(F)(F)F ; Cn1nc(NC(=O)COc2ccc(-c3cnc4ccccn34)c(F)n2)cc1C(F)(F)F]

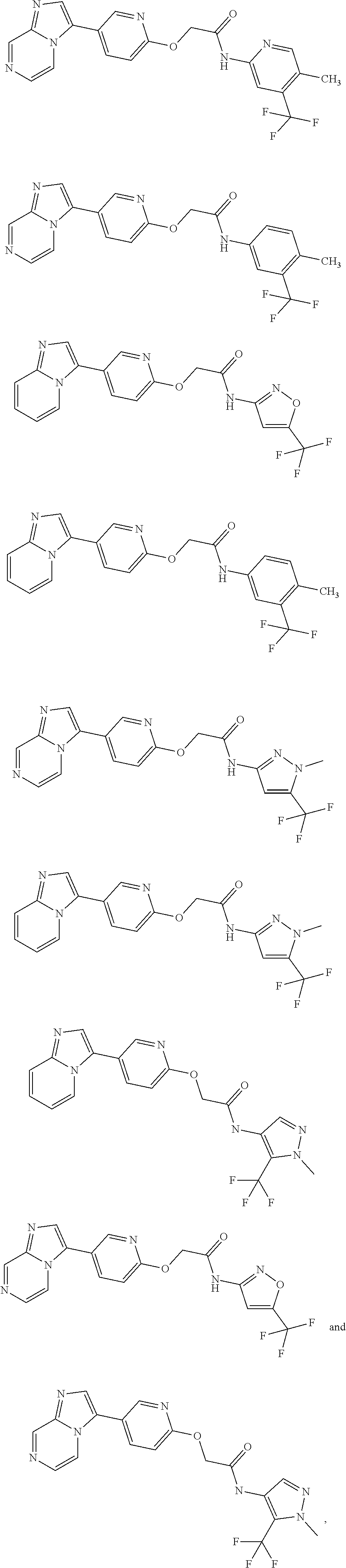

or a pharmaceutically acceptable salt, solvate or derivative thereof.

16. A compound according to any one of Clauses 1 to 13, or a pharmaceutically acceptable salt, solvate or derivative thereof, wherein L is selected from:

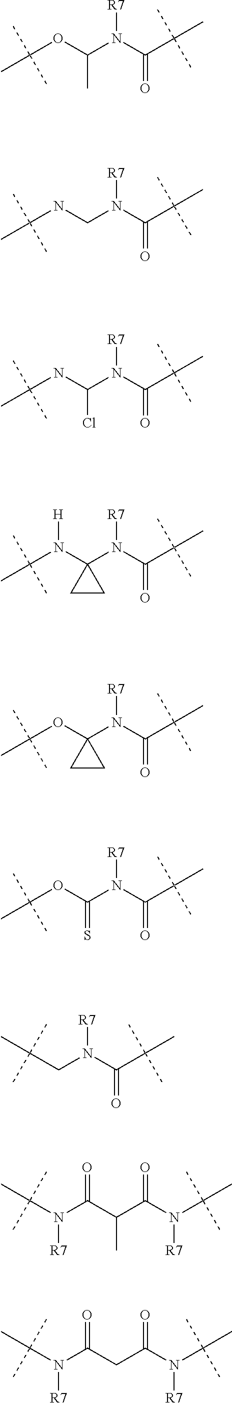

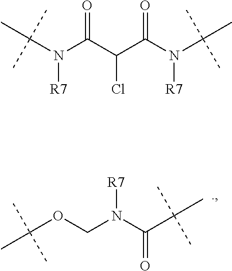

optionally wherein is selected from

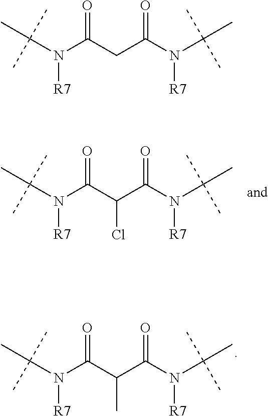

17. A compound according to any one of Clauses 1 to 13, or a pharmaceutically acceptable salt, solvate or derivative thereof, wherein L is:

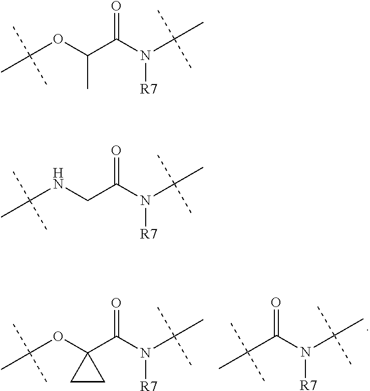

18. A compound according to Clause 1 which is selected from:

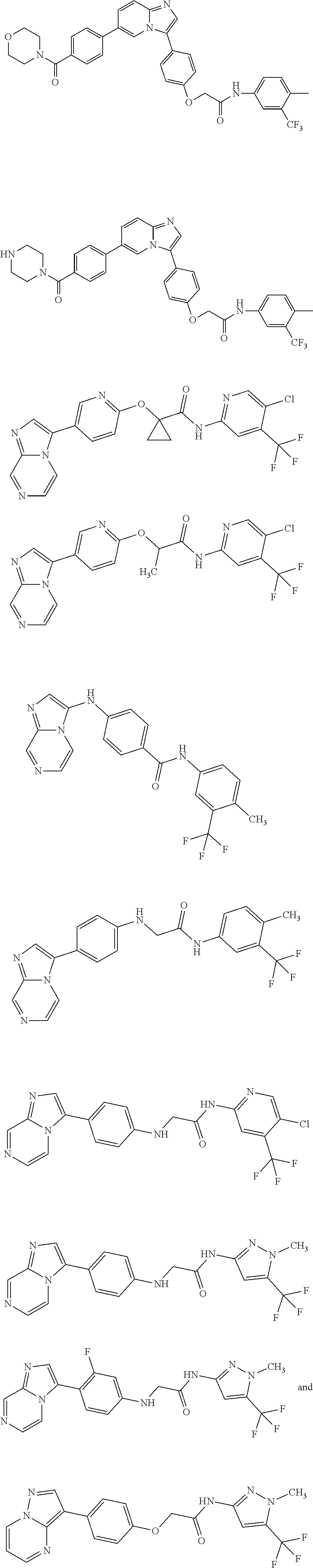

or a pharmaceutically acceptable salt, solvate or derivative thereof.

19. Use of a compound of formula I as defined in any one of Clauses 1 to 18, or a pharmaceutically acceptable salt, solvate or derivative thereof, in the manufacture of a medicament to treat one or more of macular degeneration, diabetic retinopathy, and angiogenesis.

20. A method of treating one or more of macular degeneration, diabetic retinopathy, and angiogenesis, which method comprises administering a therapeutically effective amount of a compound of formula as defined in any one of Clauses 1 to 18 or a pharmaceutically acceptable salt, solvate or derivative thereof.

21. A compound of formula I as defined in any one of Clauses 1 to 18 or a pharmaceutically acceptable salt, solvate or derivative thereof, for use in treating one or more of macular degeneration, diabetic retinopathy, and angiogenesis.

22. A pharmaceutical composition comprising a compound of formula I as defined in any one of Clauses 1 to 18 or a pharmaceutically acceptable salt, solvate or derivative thereof.

23. A compound according to Clause 1, or a pharmaceutically acceptable salt, solvate or derivative thereof, wherein:

X₁ and X₂ each independently represent N or CRₐ
Rₐ independently represents H, NH₂, halo, C₁₋₅ alkyl, C₁₋₅alkoxy, C₂₋₅ alkenyl and C₂₋₅alkynyl (which latter four groups are unsubstituted or substituted by one or more halo substituents);
A is selected from the group consisting of:

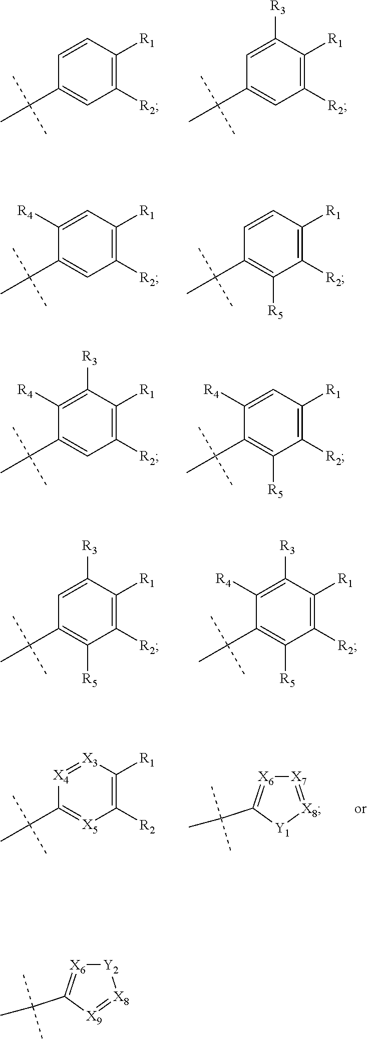

where:
the dotted line represents the point of attachment to the rest of the molecule;
each R₁ to R₅ is independently selected from halo, C₁₋₅ alkyl, C₁₋₅ alkoxy, C₂₋₅ alkenyl, C₂₋₅ alkynyl, which latter four groups are unsubstituted or substituted by one or more halo substituents;
X₃ represents N, CH or CR₃, where R₃ is as defined above, X₄ represents N, CH or CR₄, where R₄ is as defined above, X₅ represents N, CH or CR₅, where R₅ is as defined above, provided that only one or two of X₃ to X₅ is N;
each X₆ to X₉ independently represents N, CH or CR₆, where each R₈ is independently selected from C₁₋₅ alkyl, C₁₋₅ alkoxy, C₂₋₅ alkenyl, C₂₋₅ alkynyl, which four groups are unsubstituted or substituted by one or more halo substituents;
Y₁ and Y₂ each independently represent NR_{N}, O or S;
R_{N} represents H, C₁₋₅ alkyl, C₂₋₅ alkenyl, C₂₋₅ alkynyl, which latter three groups are unsubstituted or substituted by one or more halo substituents;
L is a linking group of the formula:

-M-(CR_{L}R_{M})ₐ—C(O)—NR₇—; or

-M-(CR_{L}R_{M})ₐ—NR₇—C(O)—;

where M represents a covalent bond, O or NH;
R_{L} and R_{M} each independently represent H, methyl, ethyl, fluoro or chloro, or R_{L} and R_{M} together form a C₃ or C₄ cycloalkyl ring, carbonyl or thiocarbonyl group;
a represents 0 or 1;
R₇ and R_{7′} represent H or an optionally substituted alkyl group;
Z represents a heterocycle selected from the group consisting of:

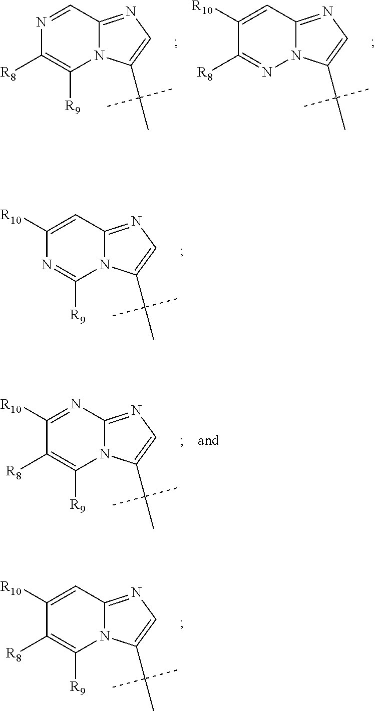

where:
the dotted line represents the point of attachment to the rest of the molecule, and Z is attached to the rest of the molecule via a covalent bond, or via a —O— or —NH— group;
each of R₈ to R₁₀ are independently selected from H, Me, C₁ to C₅ alkoxy which is unsubstituted or substituted by one or more halo groups, OC(O)R₁₁, C(O)OR₁₂, C₂ to C₅ alkynyl substituted by one or more halo groups or NR₁₃R₁₄, and one of R₈ to R₁₀ may be a

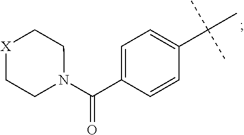

group of the formula

where X represents O or NH

R₁₁ and R₁₂ each independently represent, at each occurrence, optionally substituted alkyl;
R₁₃ and R₁₄ each independently represent, at each occurrence, H or optionally substituted alkyl;
provided that when X₁ and X₂ are both CH, L is

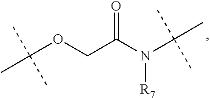

### and A is

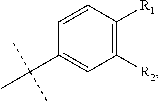

then Z is not an optionally substituted heteroaryl selected from optionally substituted tetrazolyl or optionally substituted imidazopyridinyl.
The invention also provides the following numbered statements.

1. A compound of formula I:

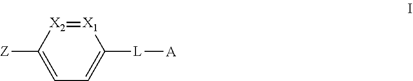

wherein:
X₁ and X₂ each independently represent N or CRₐ Rₐ independently represents H, NH₂, halo, C₁₋₅ alkyl, C₁₋₅ alkoxy, C₂₋₅ alkenyl and C₂₋₅ alkynyl (which latter four groups are unsubstituted or substituted by one or more halo substituents);
A is selected from the group consisting of:

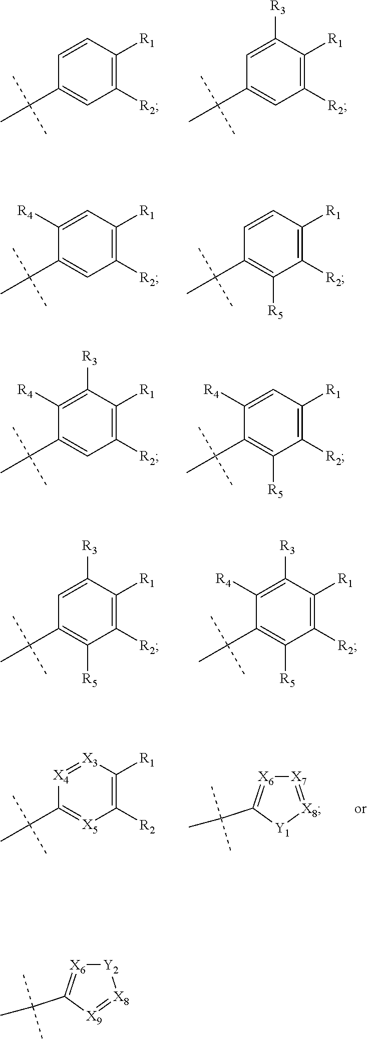

where:
the dotted line represents the point of attachment to the rest of the molecule; each R₁ to R₅ is independently selected from halo, C₁₋₅ alkyl, C₁₋₅ alkoxy, C₂₋₅ alkenyl, C₂₋₅ alkynyl, which latter four groups are unsubstituted or substituted by one or more halo substituents;
X₃ represents N, CH or CR₃, where R₃ is as defined above, X₄ represents N, CH or CR₄, where R₄ is as defined above, X₅ represents N, CH or CR₅, where R₅ is as defined above, provided that only one or two of X₃ to X₅ is N;
each X₆ to X₉ independently represents N, CH or CR₆, where each R₆ is independently selected from C₁₋₅ alkyl, C₁₋₅ alkoxy, C₂₋₅ alkenyl, C₂₋₅ alkynyl, which four groups are unsubstituted or substituted by one or more halo substituents;
Y₁ and Y₂ each independently represent NR_{N}, O or S;
R_{N} represents H, C₁₋₅ alkyl, C₂₋₅ alkenyl, C₂₋₅ alkynyl, which latter three groups are unsubstituted or substituted by one or more halo substituents;
L is a linking group of the formula:

-M-(CR_{L}R_{M})ₐ—C(O)—NR₇—; or

-M-(CR_{L}R_{M})ₐ—NR₇—C(O)—;

where M represents a covalent bond, O or NH;
R_{L} and R_{M} each independently represent H, methyl, ethyl, fluoro or chloro, or R_{L} and R_{M} together form a C₃ or C₄ cycloalkyl ring, carbonyl or thiocarbonyl group;
a represents 0 or 1;
R₇ and R_{7′} represent H or an optionally substituted alkyl group;
Z represents a heterocycle selected from the group consisting of:

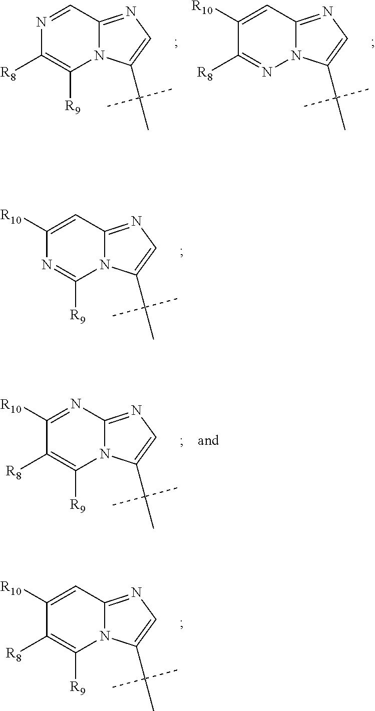

where:
the dotted line represents the point of attachment to the rest of the molecule, and Z is attached to the rest of the molecule via a covalent bond, or via a —O— or —NH— group, optionally where Z is attached to the rest of the molecule via a covalent bond;
each of R₈ to R₁₀ are independently selected from H, Me, C₁ to C₅ alkoxy which is unsubstituted or substituted by one or more halo groups, OC(O)R₁₁, C(O)OR₁₂, C₂ to C₅ alkynyl substituted by one or more halo groups or NR₁₃R₁₄, and one of R₈ to R₁₀ may be a
group of the formula

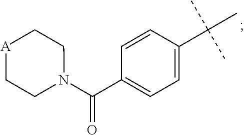

where A represents O or NH

R₁₁ and R₁₂ each independently represent, at each occurrence, optionally substituted alkyl;
R₁₃ and R₁₄ each independently represent, at each occurrence, H or optionally substituted alkyl; or
a pharmaceutically acceptable salt, solvate or derivative thereof, provided that when X₁ and X₂ are both CH, L is

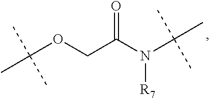

### and A is

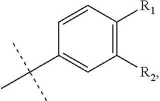

then Z is not an optionally substituted heteroaryl selected from optionally substituted tetrazolyl or optionally substituted imidazopyridinyl.

2. A compound according to Statement 1, or a pharmaceutically acceptable salt, solvate or derivative thereof, wherein Rₐ independently represents H, NH₂, F, Cl, or C₁₋₃ alkyl, which C₁₋₃ alkyl group is unsubstituted or substituted by one, two or three fluoro or chloro substituents,

preferably wherein Rₐ is H or F.

3. A compound according to Statement 1 or 2, or a pharmaceutically acceptable salt, solvate or derivative thereof, wherein:

each R₁ to R₅ independently represents halo, C₁₋₃ alkyl, C₁₋₃ alkoxy, C₂₋₃ alkenyl and C₂₋₃ alkynyl (which four groups are unsubstituted or substituted by one or more halo substituents), preferably wherein each R₁ to R₅ independently represents fluoro, chloro, methyl or ethyl, which methyl and ethyl groups may be unsubstituted or substituted by one, two or three fluoro or chloro groups.

4. A compound according to Statement 1 or 2, or a pharmaceutically acceptable salt, solvate or derivative thereof, wherein:

Y₁ and Y₂ independently represent O, NC₁₋₃ alkyl or NH; and/or
R₆ independently represents C₁₋₃ alkyl, C₁₋₃ alkoxy, C₂₋₃ alkenyl and C₂₋₃ alkynyl (which four groups are unsubstituted or substituted by one or more halo substituents),
preferably wherein Y₁ and Y₂ independently represent O, NMe or NH, and/or
R₆ independently represents fluoro, chloro, methyl or ethyl, which methyl and ethyl groups may be unsubstituted or substituted by one, two or three fluoro or chloro groups.

4. A compound according to Statement 3, or a pharmaceutically acceptable salt, solvate or derivative thereof, wherein each of R₁ to R₅ and R₆ independently represents methyl, trifluoromethyl, fluoro or chloro.

5. A compound according to any one of the preceding Statements, or a pharmaceutically acceptable salt, solvate or derivative thereof, wherein:
(a) each R₈ to R₁₀ independently represents H, Me, C₁₋₂ alkoxy which is unsubstituted or substituted by one or more halo groups, OC(O)R₁₁, C(O)OR₁₂, C₂ to C₃ alkynyl substituted by one or more halo groups, or NR₁₃R₁₄,
   R₁₁ and R₁₂ each independently represent methyl or ethyl,
   R₁₃ and R₁₄ each independently represent H, methyl or ethyl; or
(b) one of R₈ to R₁₀ represents a group of the formula

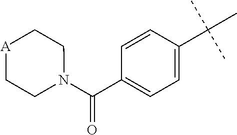

where A represents O or NH, and the remaining two of R₈ to R₁₀ are as defined in part (a).

6. A compound according to any one of the preceding Statements, or a pharmaceutically acceptable salt, solvate or derivative thereof, wherein Z represents a heterocycle selected from:

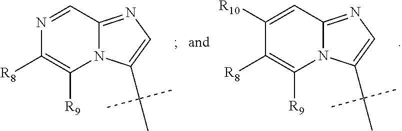

7. A compound according to any one of any one of the preceding Statements, or a pharmaceutically acceptable salt, solvate or derivative thereof, wherein A is selected from the group consisting of:

| | |
|---|---|
| 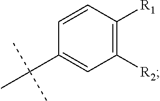 | 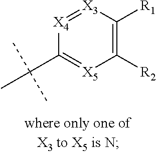 |
| 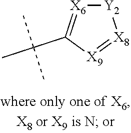 | 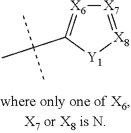 |

8. A compound according to any one of the preceding Statements, or a pharmaceutically acceptable salt, solvate or derivative thereof, wherein:

M represents O or NH; and/or
R_{L} and R_{M} each independently represent H, methyl or chloro, or R_{L} and R_{M} together represent thiocarbonyl or cyclopropyl; and/or
a represents 1.

9. A compound according to any one of the preceding Statements, or a pharmaceutically acceptable salt, solvate or derivative thereof, wherein L represents:

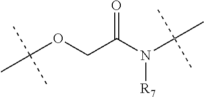

where the dotted lines represent the point of attachment to the rest of the molecule.

10. A compound according to Statement 9 which is selected from

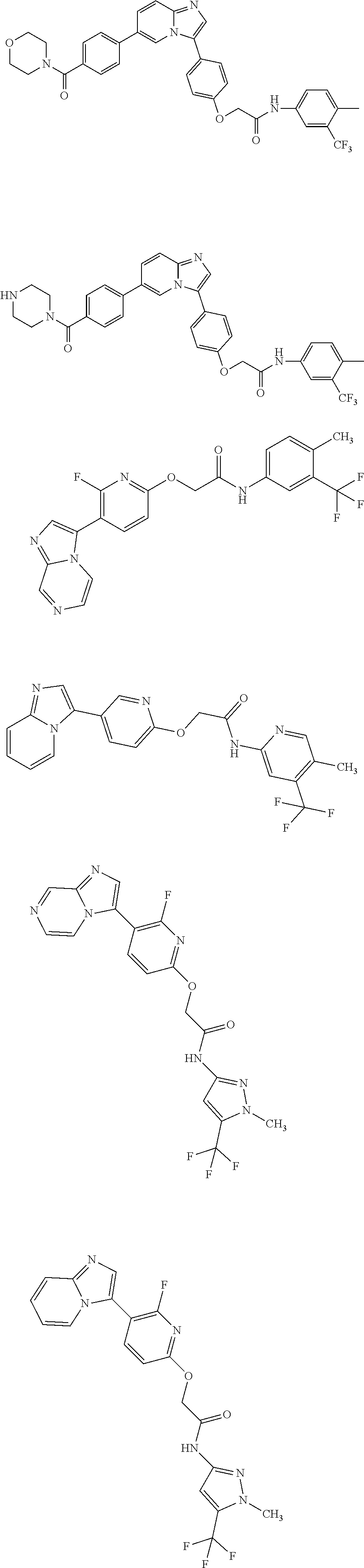 [SMILES: Cc1ccc(NC(=O)COc2ccc(-c3cnc4ccc(-c5ccc(C(=O)N6CCOCC6)cc5)cn34)cc2)cc1C(F)(F)F ; Cc1ccc(NC(=O)COc2ccc(-c3cnc4ccc(-c5ccc(C(=O)N6CCNCC6)cc5)cn34)cc2)cc1C(F)(F)F ; Cc1ccc(NC(=O)COc2ccc(-c3cnc4cnccn34)c(F)n2)cc1C(F)(F)F ; Cc1cnc(NC(=O)COc2ccc(-c3cnc4ccccn34)cn2)cc1C(F)(F)F ; Cn1nc(NC(=O)COc2ccc(-c3cnc4cnccn34)c(F)n2)cc1C(F)(F)F ; Cn1nc(NC(=O)COc2ccc(-c3cnc4ccccn34)c(F)n2)cc1C(F)(F)F]

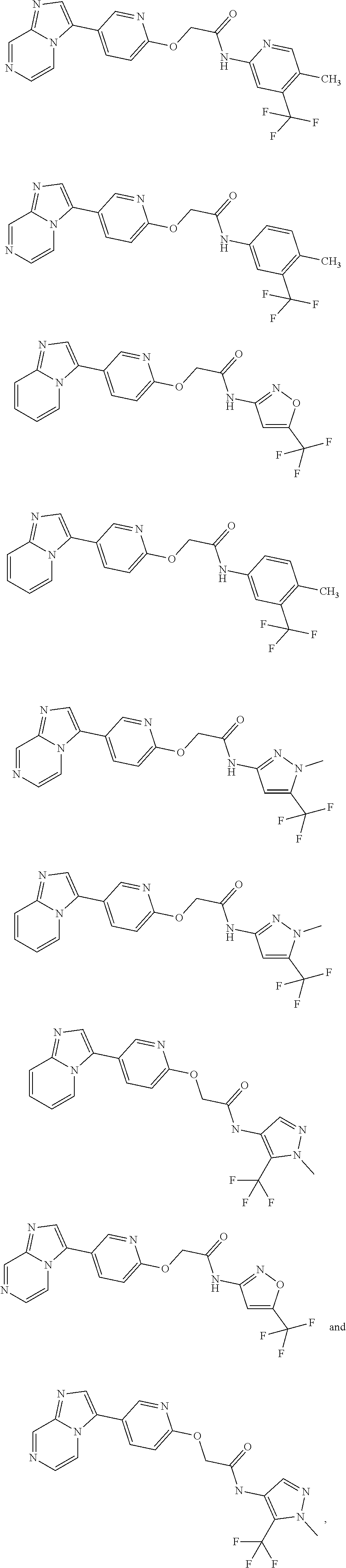

or a pharmaceutically acceptable salt, solvate or derivative thereof.

11. A compound according to any one of Statements 1 to 8, or a pharmaceutically acceptable salt, solvate or derivative thereof, wherein L is selected from:

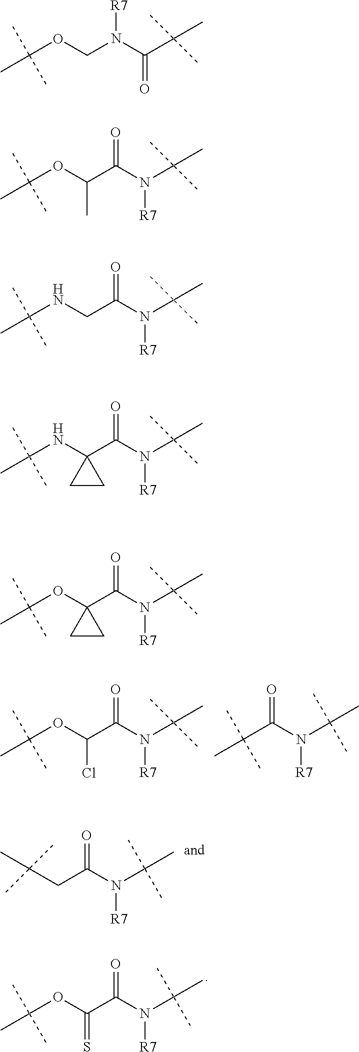

12. A compound according to Statement 11 which is selected from

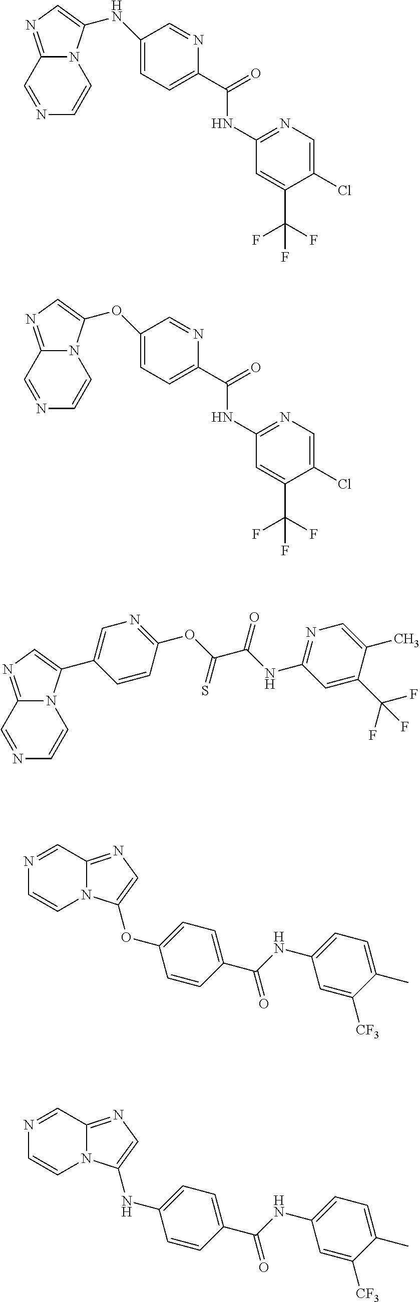 [SMILES: O=C(Nc1cc(C(F)(F)F)c(Cl)cn1)c1ccc(Nc2cnc3cnccn23)cn1 ; O=C(Nc1cc(C(F)(F)F)c(Cl)cn1)c1ccc(Oc2cnc3cnccn23)cn1 ; Cc1cnc(NC(=O)C(=S)Oc2ccc(-c3cnc4cnccn34)cn2)cc1C(F)(F)F ; Cc1ccc(NC(=O)c2ccc(Oc3cnc4cnccn34)cc2)cc1C(F)(F)F ; Cc1ccc(NC(=O)c2ccc(Nc3cnc4cnccn34)cc2)cc1C(F)(F)F]

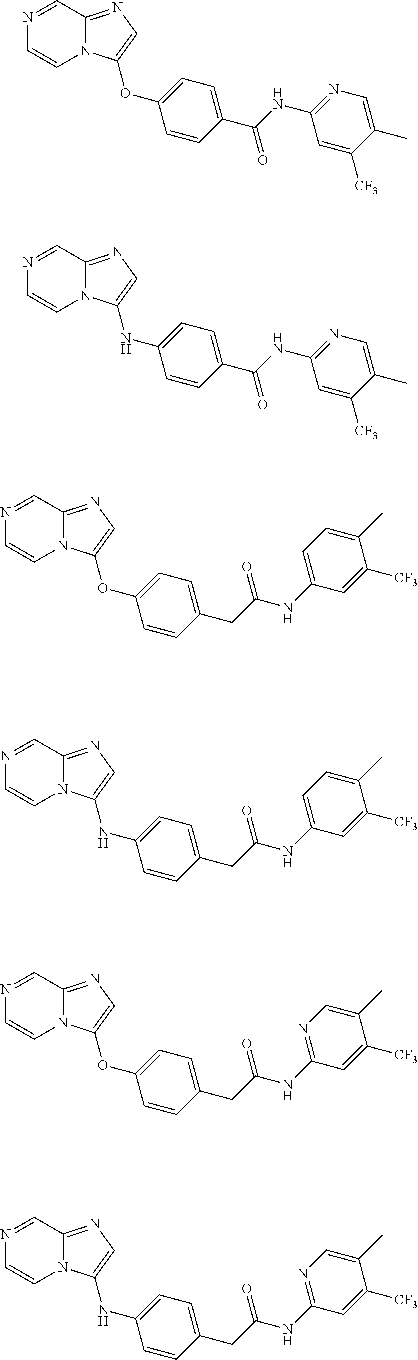 [SMILES: Cc1cnc(NC(=O)c2ccc(Oc3cnc4cnccn34)cc2)cc1C(F)(F)F ; Cc1cnc(NC(=O)c2ccc(Nc3cnc4cnccn34)cc2)cc1C(F)(F)F ; Cc1ccc(NC(=O)Cc2ccc(Oc3cnc4cnccn34)cc2)cc1C(F)(F)F ; Cc1ccc(NC(=O)Cc2ccc(Nc3cnc4cnccn34)cc2)cc1C(F)(F)F ; Cc1cnc(NC(=O)Cc2ccc(Oc3cnc4cnccn34)cc2)cc1C(F)(F)F ; Cc1cnc(NC(=O)Cc2ccc(Nc3cnc4cnccn34)cc2)cc1C(F)(F)F]

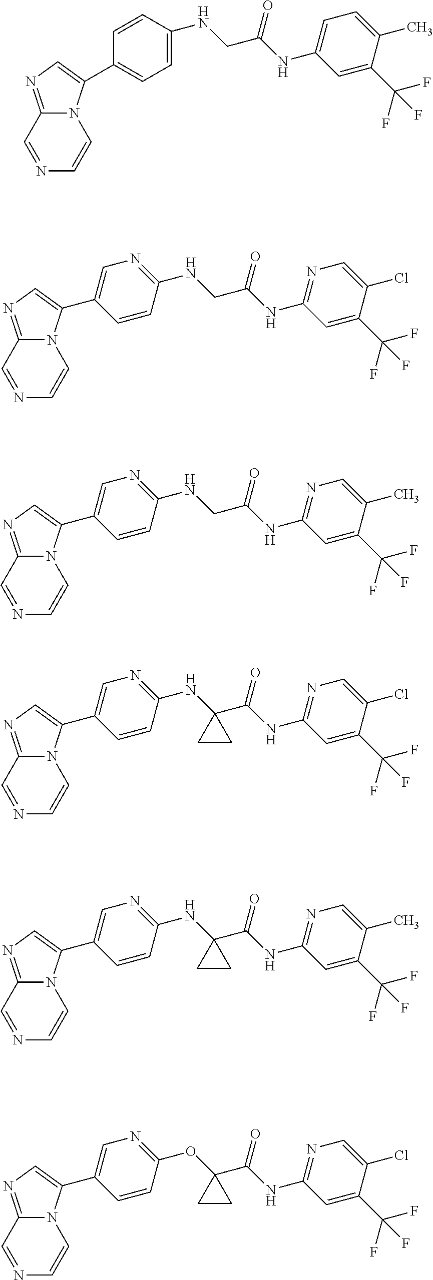 [SMILES: Cc1ccc(NC(=O)CNc2ccc(-c3cnc4cnccn34)cc2)cc1C(F)(F)F ; O=C(CNc1ccc(-c2cnc3cnccn23)cn1)Nc1cc(C(F)(F)F)c(Cl)cn1 ; Cc1cnc(NC(=O)CNc2ccc(-c3cnc4cnccn34)cn2)cc1C(F)(F)F ; O=C(Nc1cc(C(F)(F)F)c(Cl)cn1)C1(Nc2ccc(-c3cnc4cnccn34)cn2)CC1 ; Cc1cnc(NC(=O)C2(Nc3ccc(-c4cnc5cnccn45)cn3)CC2)cc1C(F)(F)F ; O=C(Nc1cc(C(F)(F)F)c(Cl)cn1)C1(Oc2ccc(-c3cnc4cnccn34)cn2)CC1]

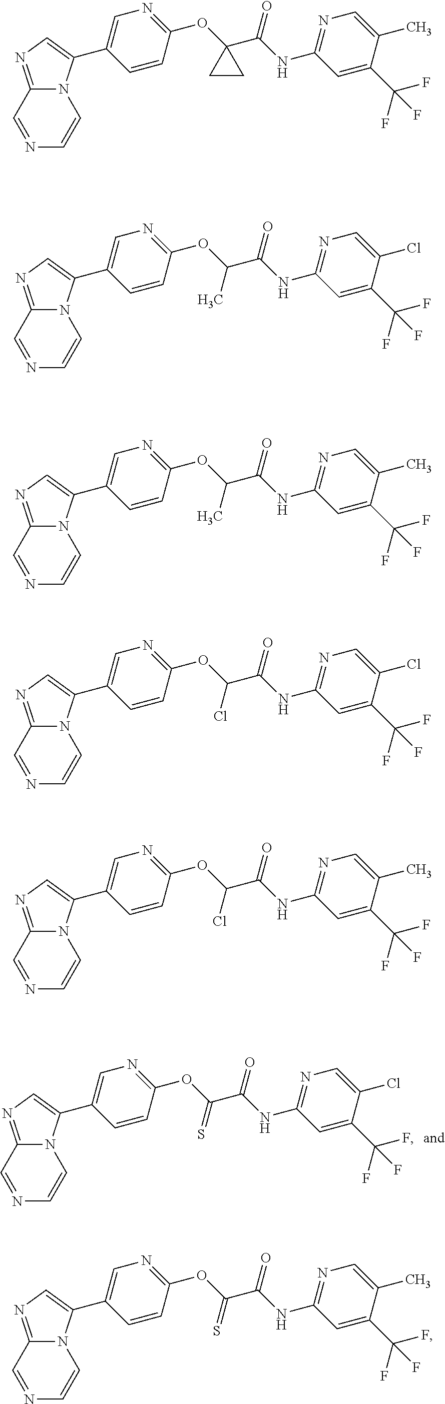

or a pharmaceutically acceptable or a salt, solvate or derivative thereof.

13. Use of a compound of formula I as defined in any one of Statements 1 to 12, or a pharmaceutically acceptable salt, solvate or derivative thereof, in the manufacture of a medicament to treat one or more of macular degeneration, diabetic retinopathy, cancer (e.g. prostate cancer, colon cancer, rectal cancer, colorectal cancer, acute myeloid leukaemia or chronic myelomonocytic leukaemia) and angiogenesis.

14. A method of treating one or more of macular degeneration, diabetic retinopathy, cancer (e.g. prostate cancer, colon cancer, rectal cancer, colorectal cancer, acute myeloid leukaemia or chronic myelomonocytic leukaemia) and angiogenesis, which method comprises administering a therapeutically effective amount of a compound of formula as defined in any one of Statements 1 to 12 or a pharmaceutically acceptable salt, solvate or derivative thereof.

15. A compound of formula I as defined in any one of Statements 1 to 12 or a pharmaceutically acceptable salt, solvate or derivative thereof, for use in treating one or more of macular degeneration, diabetic retinopathy, cancer (e.g. prostate cancer, colon cancer, rectal cancer, colorectal cancer, acute myeloid leukaemia or chronic myelomonocytic leukaemia) and angiogenesis.

16. A pharmaceutical composition comprising a compound of formula I as defined in any one of Statements 1 to 12 or a pharmaceutically acceptable salt, solvate or derivative thereof.

### DRAWINGS

FIG. 1 represents Western-blot analysis result of the compounds of Examples 1 and 2 in relation to inhibition of PDGFRβ and p-SHP2 signalling in HEK293 cells expressing PDGFRβ.

FIG. 2 illustrates the effect of the compounds of Examples 1 to 4 on Ba/F3 cells expressing PDGFR. Results for imatinib and quizartinib are provided as positive controls.

FIG. 3 illustrates the effect of the compounds of Examples 5 to 7 on Ba/F3 cells expressing FIt3 kinase. Results for imatinib (negative control) and quizartinib (positive control) are also provided.

FIG. 4 shows the ability of a compound according to the invention to prevent choroid damage in vivo.

### DESCRIPTION

It has surprisingly been found that compounds of formula I described herein, including pharmaceutically acceptable salts, solvates and derivatives thereof, are potent inhibitors of angiogenesis. The compounds of the invention are useful in the treatment of angiogenesis and angiogenesis-related diseases or disorders such as eye disorders, particularly macular degeneration (e.g. age-related macular degeneration) and diabetic retinopathy.

Thus, according to a first aspect of the invention there is provided a compound of formula I:

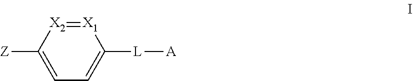

wherein:
X₁ and X₂ each independently represent N or CRₐ
Rₐ independently represents H, NH₂, halo, C₁₋₅ alkyl, C₁₋₅ alkoxy, C₂₋₅ alkenyl and C₂₋₅ alkynyl (which latter four groups are unsubstituted or substituted by one or more halo substituents);
A is selected from the group consisting of

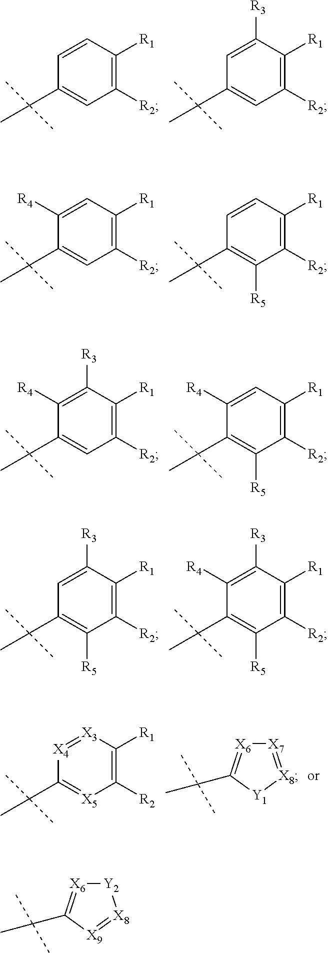

where:
the dotted line represents the point of attachment to the rest of the molecule;
each R₁ to R₅ is independently selected from halo, C₁₋₅ alkyl, C₁₋₅ alkoxy, C₂₋₅ alkenyl, C₂₋₅ alkynyl, which latter four groups are unsubstituted or substituted by one or more halo substituents;
X₃ represents N, CH or CR₃, where R₃ is as defined above, X₄ represents N, CH or CR₄, where R₄ is as defined above, X₅ represents N, CH or CR₅, where R₅ is as defined above, provided that only one or two of X₃ to X₅ is N;
each X₆ to X₆ independently represents N, CH or CR₆, where each Rₐ is independently selected from C₁₋₅ alkyl, C₁₋₅ alkoxy, C₂₋₅ alkenyl, C₂₋₅ alkynyl, which four groups are unsubstituted or substituted by one or more halo substituents;
wherein in any moiety A, one of R₁ to R₆ may be piperazine, methylpiperazine or ethylpiperazine, each of which may be connected to the rest of the moiety A via a carbon or nitrogen atom in the piperazine ring;
Y₁ represent NR_{N}, O or S;
Y₂ represents NR_{N}, NR_{Y} O or S;
R_{N} represents H, C₁₋₅ alkyl, C₂₋₅ alkenyl, C₂₋₅ alkynyl, which latter three groups are unsubstituted or substituted by one or more halo substituents;
R_{Y} represents piperazine, methylpiperazine or ethylpiperazine, each of which is connected to the nitrogen atom in Y₂ via a carbon atom in the piperazine ring;
L is a linking group of the formula:

-M-(CR_{L}R_{M})ₐ—C(O)—NR₇—;

-M-(CR_{L}R_{M})ₐ—NR₇—C(O)—; or

-M-C(O)—(CR_{N}R_{O})—C(O)-M-

where M represents a covalent bond, O or NH;
R_{L} and R_{M} each independently represent H, methyl, ethyl, fluoro or chloro, or R_{L} and R_{M} together form a C₃ or C₄ cycloalkyl ring, carbonyl or thiocarbonyl group;
a represents 0 or 1;
R₇ and R_{7′} represent H or an optionally substituted alkyl group;
R_{N} and R_{O} each independently represent H, methyl, ethyl, fluoro or chloro;
Z represents a heterocycle selected from the group consisting of:

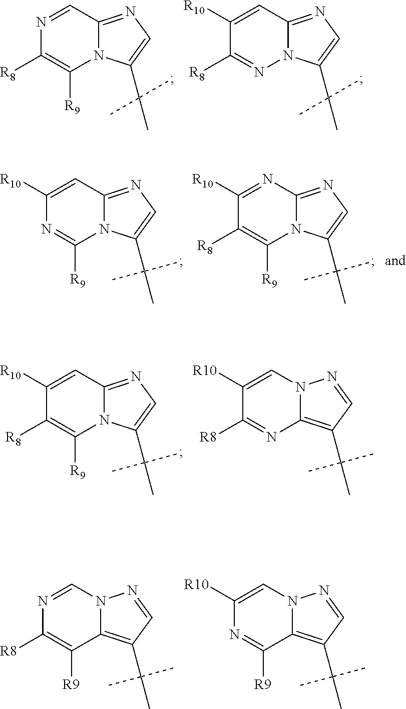

where:
the dotted line represents the point of attachment to the rest of the molecule, and Z is attached to the rest of the molecule via a covalent bond, or via a —O— or —NH— group;
each of R₆ to R₁₀ are independently selected from H, hydroxy, C₁ to C₅ alkyl, C₁ to C₅ alkoxy, (which latter two groups are unsubstituted or substituted by one or more halo groups), OC(O)R₁₁, C(O)OR₁₂, C₂ to C₅ alkynyl (which is unsubstituted or substituted by one or more halo groups) or NR₁₃R₁₄, and O—(C₁₋₄ alkyleneyl)-O—C₁₋₄ alkyl, and one of R₆ to R₁₀ may be a group of the formula:

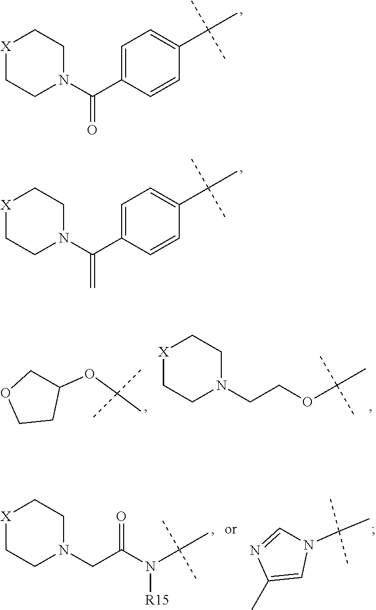

where X represents O, NR_{X},

R_{X} represents H or C₁₋₄alkyl,
R₁₁ and R₁₂ each independently represent, at each occurrence, optionally substituted alkyl;
R₁₃ and R₁₄ each independently represent, at each occurrence, H or optionally substituted alkyl;
R₁₅ represents H or C₁₋₂ alkyl; or
a pharmaceutically acceptable salt, solvate or derivative thereof, provided that when X₁ and X₂ are both CH, L is

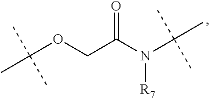

### and A is

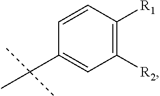

then Z is not an optionally substituted heteroaryl selected from optionally substituted tetrazolyl or optionally substituted imidazopyridinyl.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like. As used herein, C₁-C₅ alkyl refers to an alkyl group having 1 to 5 carbon atoms.

“Alkylene” refers to divalent alkyl groups preferably having from 1 to 10 carbon atoms and more preferably 1 to 6 carbon atoms. Examples of such alkylene groups include methylene (—CH₂—), ethylene (—CH₂CH₂—), and the propylene isomers (e.g., —CH₂CH₂CH₂— and —CH(CH₃)CH₂—), and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like. As used herein, C₂-C₅ alkylenyl refers to an alkylenyl group having 2 to 5 carbon atoms.

“Alkynyl” refers to alkynyl groups preferably having from 2 to 10 carbon atoms and more preferably 2 to 6 carbon atoms and having at least 1, and preferably from 1-2, carbon to carbon, triple bonds. Examples of alkynyl groups include ethynyl (—C≡CH), propargyl (—CH₂C≡CH), pent-2-ynyl (—CH₂C≡CCH₂—CH₃), and the like. As used herein, C₂-C₅ alkynyl refers to an alkynyl group having 2 to 5 carbon atoms.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like. As used herein, C₁-C₅ alkoxy refers to an alkoxy group having 1 to 5 carbon atoms.

“Halo” or “halogen” refers to fluoro, chloro, bromo and iodo.

“Haloalkyl” refers to an alkyl group wherein the alkyl group is substituted by one or more halo group as described above. The terms “haloalkenyl”, “haloalkynyl” and “haloalkoxy” are likewise defined.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Hückel criteria for aromaticity (ie. contains 4n+2 π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

When a moiety is disclosed herein as being substituted buy “one or more” groups, said moiety maybe substituted by one or more groups, such as one, two or three groups. In certain embodiments of the invention, said moiety may be substituted by one or two groups. In certain embodiments of the invention, said moiety may be substituted by a single group.

References herein (in any aspect or embodiment of the invention) to compounds of formula I includes references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g.(+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

“Pharmaceutically functional derivatives” of compounds of formula I as defined herein includes ester derivatives and/or derivatives that have, or provide for, the same biological function and/or activity as any relevant compound of the invention. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxyl or carbonyl group in a compound of formula I is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. 1-92, Elsevier, New York-Oxford (1985).

Compounds of formula I, as well as pharmaceutically acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

The compound of formula I in the above-mentioned aspect of the invention may be utilised in a method of medical treatment. Thus, according to further aspects of the invention, there is provided:
(a) a compound of formula I for use in medicine;
(b) a compound of formula I for use in the treatment or prevention of angiogenesis or an angiogenesis-related disease or disorder, particularly macular degeneration and diabetic retinopathy;
(c) use of a compound of formula I for the preparation of a medicament for the treatment of angiogenesis or an angiogenesis-related disease or disorder, particularly macular degeneration and diabetic retinopathy; and
(d) a method of treatment of angiogenesis or an angiogenesis-related disease or disorder, particularly macular degeneration and diabetic retinopathy, which method comprises the administration of an effective amount of a compound of formula I.

Compounds of formula I include a linker group of formula:

-M-(CR_{L}R_{M})ₐ—C(O)—NR₇—;

-M-(CR_{L}R_{M})ₐ—NR₇—C(O)—; or

-M-C(O)—(CR_{N}R_{O})—C(O)-M-

where M represents a covalent bond, O or NH;
R_{L} and R_{M} each independently represent H, methyl, ethyl, fluoro or chloro, or R_{L} and R_{M} together form a C₃ or C₄ cycloalkyl ring, carbonyl or thiocarbonyl group;
a represents 0 or 1;
R₇ and R_{7′} represent H or an optionally substituted alkyl group;
R_{N} and R_{O} each independently represent H, methyl, ethyl, fluoro or chloro;

For the avoidance of doubt, the linker may be oriented in each of the two possible configurations, i.e. either end of the linker may be attached to moiety A or the six-membered ring containing X₁ and X₂. In some embodiments of the invention, the left hand part of the linker as drawn herein is attached to the six-membered ring containing X₁ and X₂. In other embodiments of the invention the left hand part of the linker is attached to moiety A.

In some embodiments of the invention, the linker group L may be of formula:

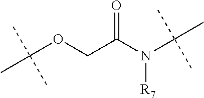

Compounds of formula I having such linkers are believed to be particularly suitable for treating eye diseases or disorders related to angiogenesis, for example macular degeneration (e.g. AMD) and diabetic retinopathy.

Other linker groups useful in compounds of formula I include linkers having the following formula:

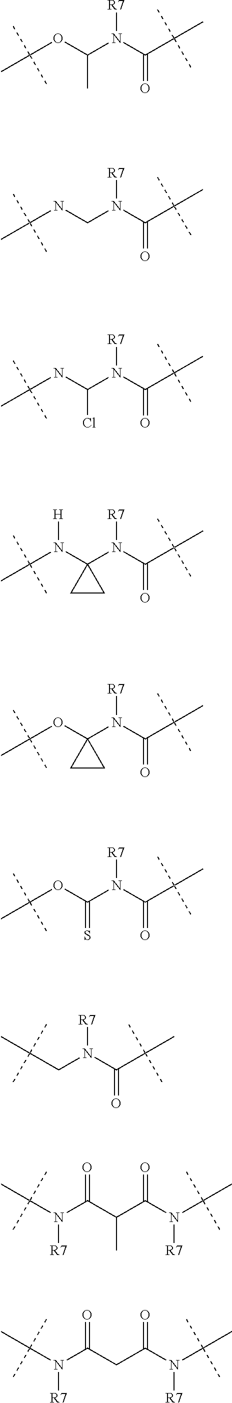

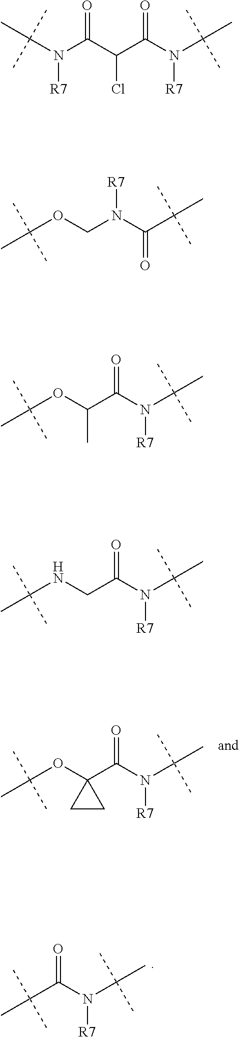

In some embodiments of the invention, L is a linker group having one of the below formula:

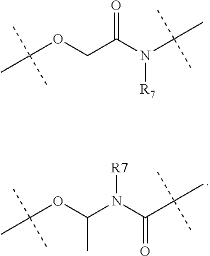

In some embodiments of the invention, the linker is selected from linkers having one of the below formula:

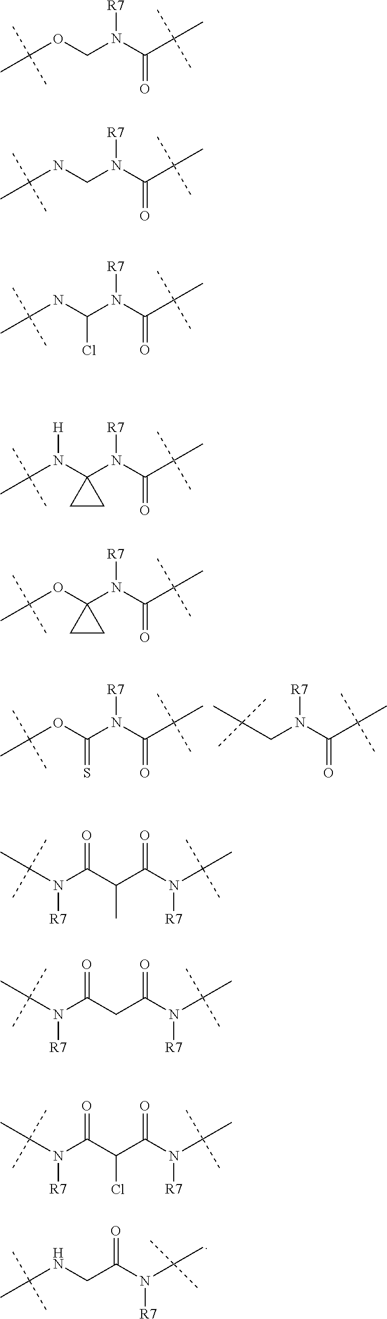

X₁ and X₂ each independently represent N or CRₐ, where Rₐ independently represents H, NH₂, halo, C₁₋₅ alkyl, C₁₋₅ alkoxy, C₂₋₅ alkenyl and C₂₋₅ alkynyl (which latter four groups are unsubstituted or substituted by one or more halo substituents). In some embodiments of the invention, Rₐ independently represents H, NH₂, F, Cl, or C₁₋₃ alkyl, which C₁₋₃ alkyl group is unsubstituted or substituted by one, two or three fluoro or chloro substituents. In particular embodiments of the invention, Rₐ is H or F.

In some embodiments of the invention, X₁ is selected from N and CH, and X₂ is selected from CH and CF.

A is selected from the group consisting of

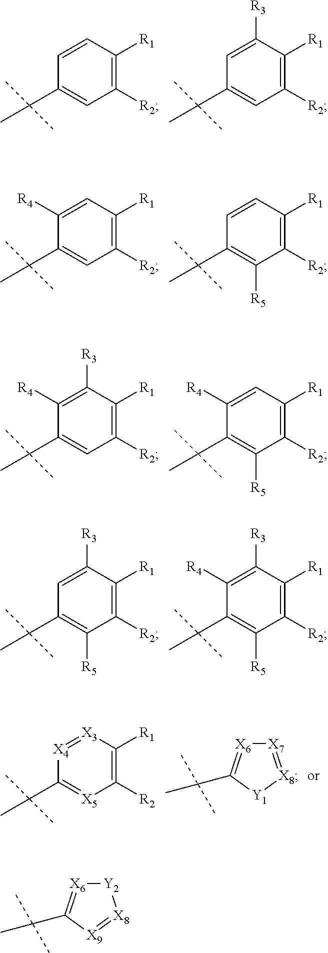

where for each moiety A:
the dotted line represents the point of attachment to the rest of the molecule;
each R₁ to R₅ is independently selected from halo, C₁₋₅ alkyl, C₁₋₅ alkoxy, C₂₋₅ alkenyl, C₂₋₅ alkynyl, which latter four groups are unsubstituted or substituted by one or more halo substituents;
X₃ represents N, CH or CR₃, where R₃ is as defined above, X₄ represents N, CH or CR₄, where R₄ is as defined above, X₅ represents N, CH or CR₅, where R₅ is as defined above, provided that only one or two of X₃ to X₅ is N;
each X₆ to X₉ independently represents N, CH or CR₆, where each R₆ is independently selected from C₁₋₅ alkyl, C₁₋₅ alkoxy, C₂₋₅ alkenyl, C₂₋₅ alkynyl, which four groups are unsubstituted or substituted by one or more halo substituents;
wherein in any moiety A, one of R₁ to R₆ may be piperazine, methylpiperazine or ethylpiperazine, each of which may be connected to the rest of the moiety A via a carbon or nitrogen atom in the piperazine ring.

In some embodiments of the invention, each R₁ to R₅ independently represents halo, C₁₋₅ alkyl, C₁₋₃ alkoxy, C₂₋₃ alkenyl and C₂₋₃ alkynyl (which four groups are unsubstituted or substituted by one or more halo substituents). In some embodiments of the invention, each R₁ to R₅ independently represents fluoro, chloro, methyl or ethyl, which methyl and ethyl groups may be unsubstituted or substituted by one, two or three fluoro or chloro groups.

In some embodiments of the invention, each R₆ independently represents C₁₋₃ alkyl, C₁₋₃ alkoxy, C₂₋₃ alkenyl and C₂₋₃ alkynyl (which four groups are unsubstituted or substituted by one or more halo substituents. In some embodiments of the invention, each R₆ independently represents fluoro, chloro, methyl or ethyl, which methyl and ethyl groups may be unsubstituted or substituted by one, two or three fluoro or chloro groups.

In some embodiments of the invention, each of R₁ to R₅ and R₆ independently represents methyl, trifluoromethyl, fluoro or chloro.

In some embodiments of the invention, R₁ represents piperazine, methylpiperazine or ethylpiperazine, each of which may be connected to the rest of the moiety A via a carbon or nitrogen atom in the piperazine ring, and R₂ to R₆ do not represent piperazine, methylpiperazine or ethylpiperazine.

Y₁ represent NR_{N}, O or S;

Y₂ represents NR_{N}, NR_{Y} O or S;

R_{N} represents H, C₁₋₅ alkyl, C₂₋₅ alkenyl, C₂₋₅ alkynyl, which latter three groups are unsubstituted or substituted by one or more halo substituents;

R_{Y} represents piperazine, methylpiperazine or ethylpiperazine, each of which is connected to the nitrogen atom in Y₂ via a carbon atom in the piperazine ring.

In some embodiments, Y₁ and Y₂ each independently represent NR_{N}, O or S, where R_{N} represents H, C₁₋₅ alkyl, C₂₋₅ alkenyl, C₂₋₅ alkynyl, which latter three groups are unsubstituted or substituted by one or more halo substituents. In some embodiments of the invention, Y₁ and Y₂ independently represent O, NC₁₋₃ alkyl or NH. In some embodiments of the invention, Y₁ and Y₂ independently represent O, NMe or NH.

In some embodiments of the invention Y₁ and Y₂ are not S.

In some embodiments of the invention in which R_{Y} represents piperazine, methylpiperazine or ethylpiperazine, the piperazine, methylpiperazine or ethylpiperazine moiety is bonded to the nitrogen atom in Y₂ via a carbon atom in the piperazine ring.

In some embodiments of the invention, A is selected from the group consisting of:

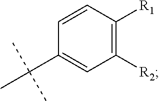

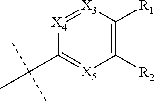

where only one of X₃ to X₅ is N;

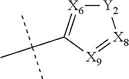

where only one of X₆, X₈ or X₉ is N; or

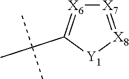

where only one of X₆, X₇ or X₈ is N.

In some embodiments of the invention, A is selected from the group consisting of:

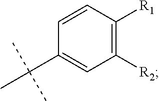

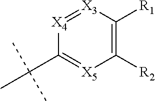

where X₃ and X₅ are CH, and X₄ is CH or N;

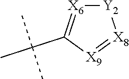

where only one of X₆ is N, X₈ and X₉ are
CH, and Y₂ is O, NR_{N} or NR_{Y}; or

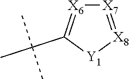

where only one of X₆, X₇ or X₈ is N and the
other two of X₆, X₇ and X₈ are CH, where Y
is as defined above.

In some embodiments of the invention, A is selected from:

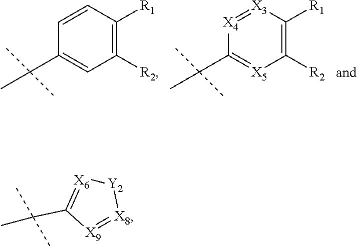

where
R₁ is selected from methyl and chlorine;
R₂ is CF₃;
X₃ and X₅ are CH;
X₄ is N;
X₆ is N;
Y₂ is O or N—CH₃;
X₆ is C—CF₃; and

X₉ is CH.

Z represents a heterocycle selected from the group consisting of:

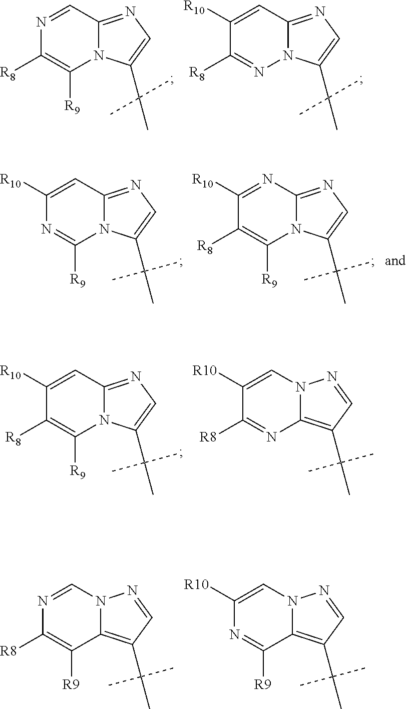

where in each moiety Z:
the dotted line represents the point of attachment to the rest of the molecule, and Z is attached to the rest of the molecule via a covalent bond, or via a —O— or —NH— group;
each of R₈ to R₁₀ are independently selected from H, hydroxy, C₁ to C₅ alkyl, C₁ to C₅ alkoxy (which latter two groups are unsubstituted or substituted by one or more halo groups), OC(O)R₁₁, C(O)OR₁₂, C₂ to C₅ alkynyl (which is unsubstituted or substituted by one or more halo groups) or NR₁₃R₁₄, and O—(C₁₋₄ alkyleneyl)-O—C₁₋₄ alkyl, and one of R₈ to R₁₀ may be a group of the formula:

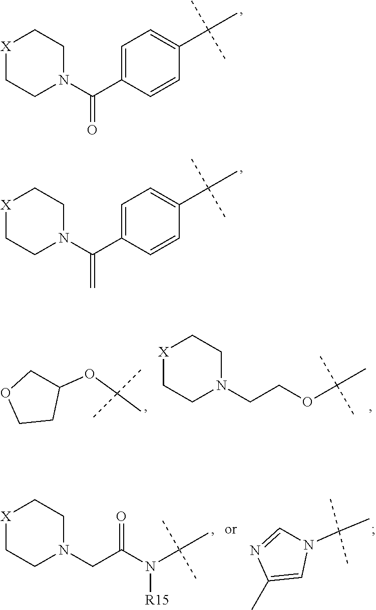

where X represents O, NR_{X},
R_{X} represents H or C₁₋₄alkyl,
R₁₁ and R₁₂ each independently represent, at each occurrence, optionally substituted alkyl;
R₁₃ and R₁₄ each independently represent, at each occurrence, H or optionally substituted alkyl;
R₁₅ represents H or C₁₋₂alkyl.

In any embodiment of the invention, the alkyl group for each of R₈ to R₁₀ may be a methyl group.

In any embodiment of the invention described herein, the C₂ to C₅ alkynyl group for each of R₈ to R₁₀ may be a C₂ to C₅ alkynyl group which is substituted by one or more halo groups.

In some embodiments of the invention, Z represents a heterocycle selected from the group consisting of

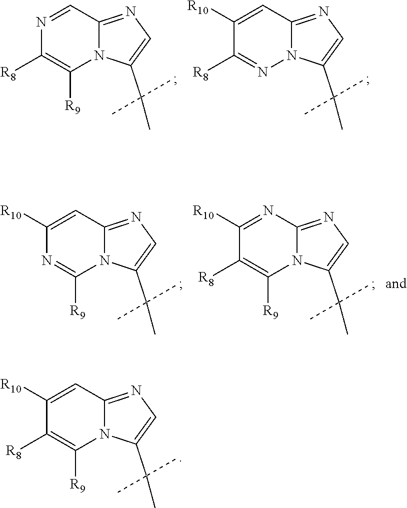

In some embodiments of the invention, Z represents a heterocycle selected from:

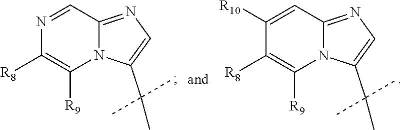

In some embodiments of the invention, Z represents a heterocycle selected from:

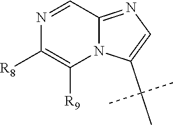

where R₈ and R₉ are H; and

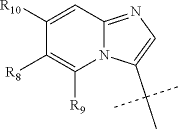

where R₉ and
R₁₀ are H, and R₈ is as defined in any
embodiment disclosed herein, for example R₈
is selected from H and

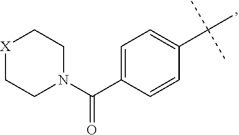

where X is O or NH.

In some embodiments of the invention, each R₈ to R₁₀ independently represents H, hydroxy, Me, C₁₋₂ alkoxy (which is unsubstituted or substituted by one or more halo groups), OC(O)R₁₁, C(O)OR₁₂, C₂ to C₃ alkynyl (which is substituted by one or more halo groups), O—(C₁₋₂ alkyleneyl)-O—C₁₋₂ alkyl, or NR₁₃R₁₄, where
R₁₁ and R₁₂ each independently represent methyl or ethyl,
R₁₃ and R₁₄ each independently represent H, methyl or ethyl.

In other embodiments of the invention, one of R₈ to R₁₀ represents a group of the formula

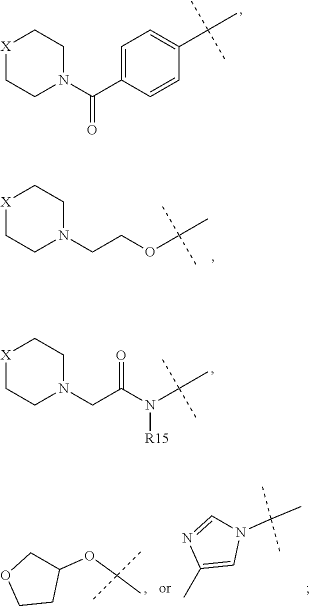

where X represents O, NH, or N—C₁₋₂ alkyl,
R₁₅ represents methyl,
and the remaining two of R₈ to R₁₀ are as defined above.

In some embodiments of the invention, each of R₈ to R₁₀ are independently selected from H, hydroxy, Me, C₁ to C₅ alkoxy (which is unsubstituted or substituted by one or more halo groups), OC(O)R₁₁, C(O)OR₁₂, C₂ to C₅ alkynyl (which is substituted by one or more halo groups) or NR₁₃R₁₄, and O—(C₁₋₄ alkyleneyl)-O—C₁₋₄ alkyl, or one of R₈ to R₁₀ may be a group of the formula:

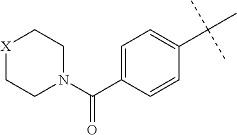

and the remaining two of R₈ to R₁₀, as well as R₁₁ to R₁₄, are as defined above.

In some embodiments of the invention, each R₈ to R₁₀ are independently selected from H, Me, C₁ to C₅ alkoxy which is unsubstituted or substituted by one or more halo groups, OC(O)R₁₁, C(O)OR₁₂, C₂ to C₅ alkynyl (which is substituted by one or more halo groups) or

NR₁₃R₁₄, and one of R₆ to R₁₀ may be a group of the formula

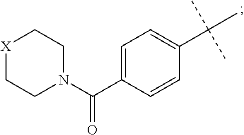

where:
X represents O or NH
R₁₁ and R₁₂ each independently represent, at each occurrence, optionally substituted alkyl;
R₁₃ and R₁₄ each independently represent, at each occurrence, H or optionally substituted alkyl.

In some embodiments of the invention, R₉ and R₁₀ (when present) are H. Thus, when one of R₈ to R₁₀ represents a group of the formula

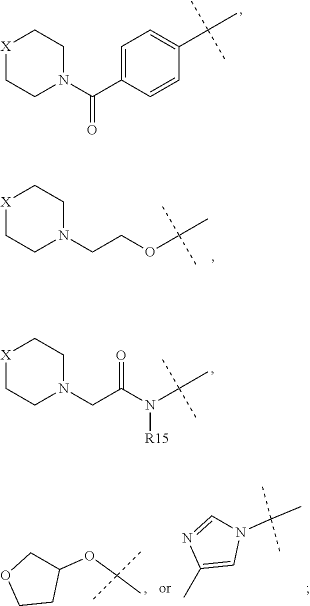

for example when one of R₈ to R₁₀ may be a group of the formula

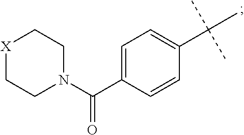

this group may be present as R₈, and R₉ and R₁₀ (when present) may be H.

In some embodiments of the invention, Z is linked to the rest of the molecule via a covalent bond.

In some embodiments of the invention, M represents O or NH. In other embodiments of the invention M represents a covalent bond.

In some embodiments of the invention, when Z is linked to the rest of the molecule via a covalent bond, then M is —O— or —NH—. In some embodiments of the invention, when Z is linked to the rest of the molecule via a —O— or —NH— group, then M is a covalent bond.

In some embodiments of the invention, R_{L} and R_{M} each independently represent H, methyl or chloro, or R_{L} and R_{M} together represent thiocarbonyl or cyclopropyl.

In some embodiments of the invention a represents 1.

In some embodiments of the invention, R₇ and R_{7′} represent H.

In formula I, when X₁ and X₂ are both CH, L is

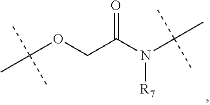

### and A is

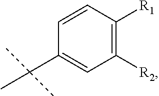

then Z is not an optionally substituted heteroaryl selected from optionally substituted tetrazolyl or optionally substituted imidazopyridinyl.

The invention also provides the following compounds, as well as pharmaceutically acceptable salts solvates and derivatives thereof.

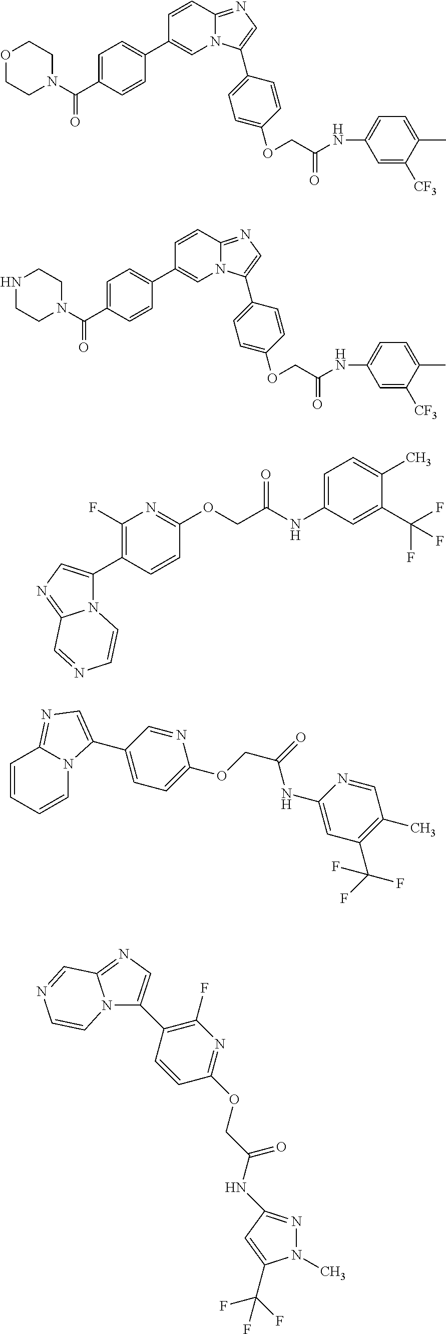

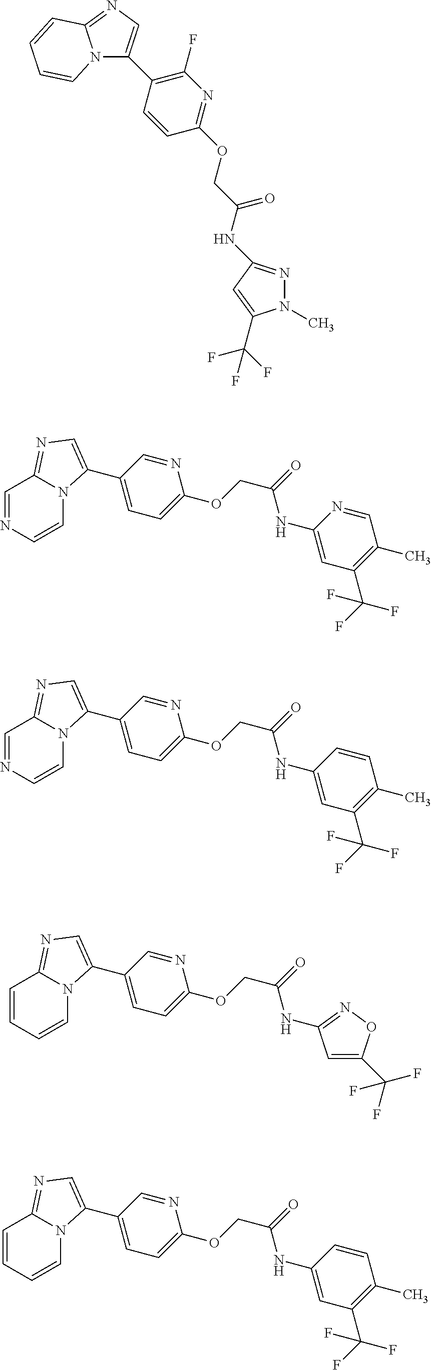 [SMILES: Cn1nc(NC(=O)COc2ccc(-c3cnc4ccccn34)c(F)n2)cc1C(F)(F)F ; Cc1cnc(NC(=O)COc2ccc(-c3cnc4cnccn34)cn2)cc1C(F)(F)F ; Cc1ccc(NC(=O)COc2ccc(-c3cnc4cnccn34)cn2)cc1C(F)(F)F ; O=C(COc1ccc(-c2cnc3ccccn23)cn1)Nc1cc(C(F)(F)F)on1 ; Cc1ccc(NC(=O)COc2ccc(-c3cnc4ccccn34)cn2)cc1C(F)(F)F]

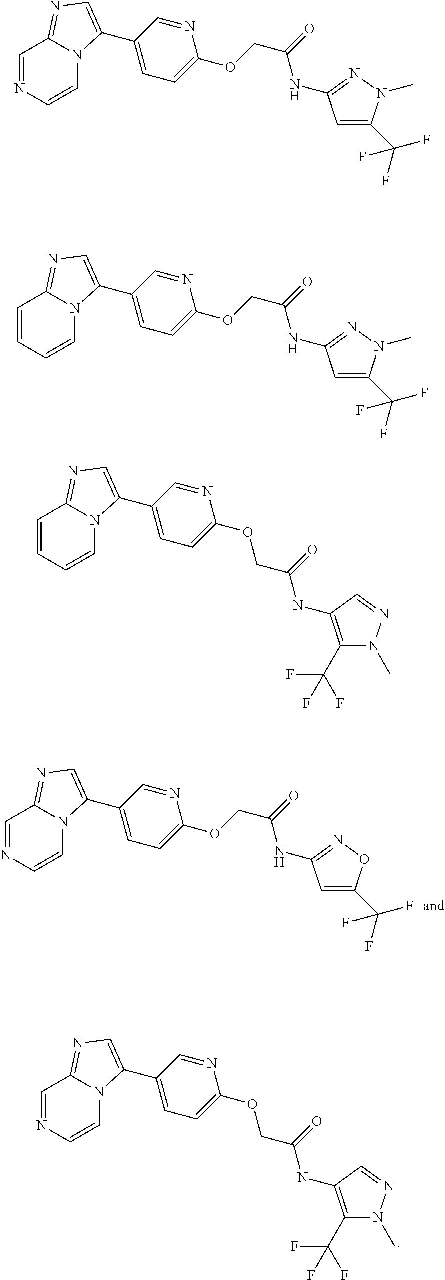 [SMILES: Cn1nc(NC(=O)COc2ccc(-c3cnc4cnccn34)cn2)cc1C(F)(F)F ; Cn1nc(NC(=O)COc2ccc(-c3cnc4ccccn34)cn2)cc1C(F)(F)F ; Cn1ncc(NC(=O)COc2ccc(-c3cnc4ccccn34)cn2)c1C(F)(F)F ; O=C(COc1ccc(-c2cnc3cnccn23)cn1)Nc1cc(C(F)(F)F)on1 ; Cn1ncc(NC(=O)COc2ccc(-c3cnc4cnccn34)cn2)c1C(F)(F)F]

The invention also provides the following compounds, as well as pharmaceutically acceptable salts, solvates and derivatives thereof.

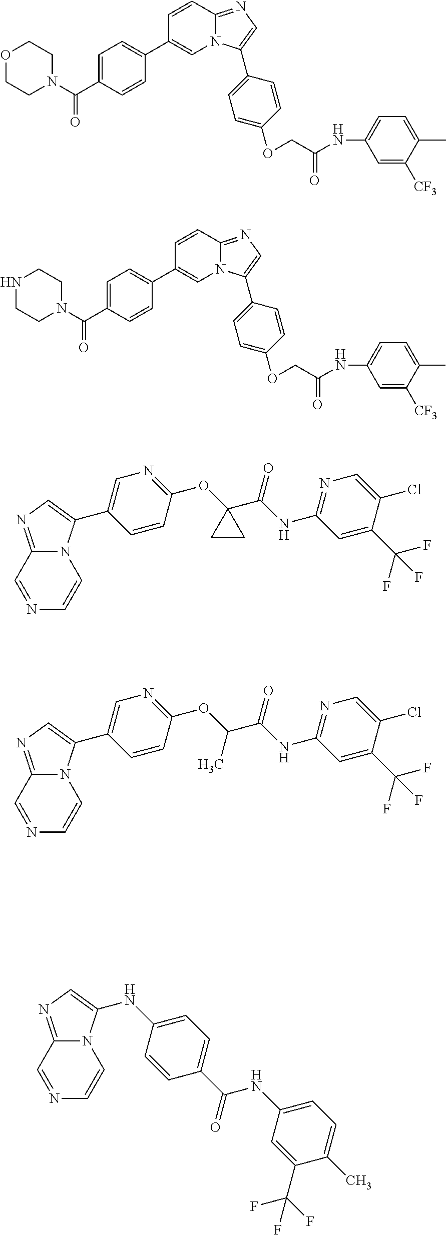 [SMILES: Cc1ccc(NC(=O)COc2ccc(-c3cnc4ccc(-c5ccc(C(=O)N6CCOCC6)cc5)cn34)cc2)cc1C(F)(F)F ; Cc1ccc(NC(=O)COc2ccc(-c3cnc4ccc(-c5ccc(C(=O)N6CCNCC6)cc5)cn34)cc2)cc1C(F)(F)F ; O=C(Nc1cc(C(F)(F)F)c(Cl)cn1)C1(Oc2ccc(-c3cnc4cnccn34)cn2)CC1 ; CC(Oc1ccc(-c2cnc3cnccn23)cn1)C(=O)Nc1cc(C(F)(F)F)c(Cl)cn1 ; Cc1ccc(NC(=O)c2ccc(Nc3cnc4cnccn34)cc2)cc1C(F)(F)F]

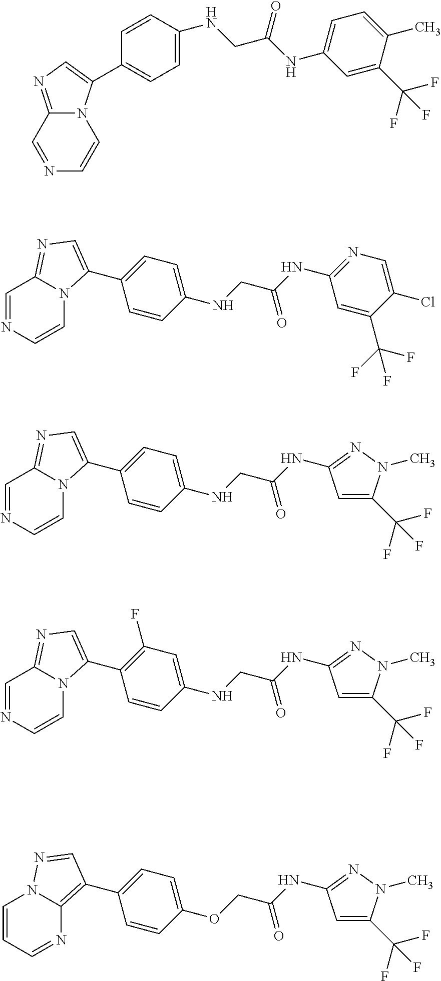 [SMILES: Cc1ccc(NC(=O)CNc2ccc(-c3cnc4cnccn34)cc2)cc1C(F)(F)F ; O=C(CNc1ccc(-c2cnc3cnccn23)cc1)Nc1cc(C(F)(F)F)c(Cl)cn1 ; Cn1nc(NC(=O)CNc2ccc(-c3cnc4cnccn34)cc2)cc1C(F)(F)F ; Cn1nc(NC(=O)CNc2ccc(-c3cnc4cnccn34)c(F)c2)cc1C(F)(F)F ; Cn1nc(NC(=O)COc2ccc(-c3cnn4cccnc34)cc2)cc1C(F)(F)F]

In this specification, the term “macular degeneration” or “MD” is intended to include age-related macular degeneration (AMD) but does not exclude macular degeneration in patients who are not elderly. Accordingly, AMD and MD as referred to herein may be used interchangeably. MD is a disease that affects a special layer of cells in the eye called the retinal pigment epithelium. This layer of cells is underneath the retina. The retinal pigment epithelium (RPE) is like a wall or barrier and is responsible for passing oxygen, sugar and other essentials up to the retina and moving waste products down to the blood vessels underneath (these vessels are called the choroid). The RPE also acts as a barrier between the choroid and the retina. When RPE cells die, the retinal cells above them also die, leading to patches of ‘missing’ retina. This is commonly called geographic atrophy or “dry” MD, which is a slow form of the disease that causes a gradual loss of vision. “Wet” macular degeneration occurs when the RPE cells fail to stop choroidal blood vessels from growing under the retina. This growth is called choroidal neovascularisation or CNV. The rapidly growing vessels are fragile with leaky walls and they ooze fluid and blood under the retina. This leads to scarring and severe loss of central vision, which if left untreated, becomes permanent. In the context of the present invention it will be appreciated that the term “macular degeneration” particularly refers to “wet” MD also known as neovascular or exudative AMD.

As used herein, the term “diabetic retinopathy” refers to a microvascular complication of diabetes. This complication can occur in the eye. Accordingly, “diabetic retinopathy” is intended to include all categories and classification, for example the earlier stage of nonproliferative diabetic retinopathy (NPDR) and the advanced stage of proliferative diabetic retinopathy (PDR) associated with abnormal blood vessel growth. Diabetic macular edema (DME) is also included within its scope. DME is a manifestation of diabetic retinopathy that occurs across all severity levels of both NPDR and PDR and represents the most common cause of vision loss in patients. DME arises from diabetes-induced breakdown of the blood-retinal barrier (BRB), with consequent vascular leakage of fluid and circulating proteins into the neural retina. The extravasation of fluid into the neural retina leads to abnormal retinal thickening and often cystoid edema of the macula.

In wet AMD and diabetic retinopathy, VEGFα is believed to play a significant role in the formation of blood vessels that grow abnormaly and leak beneath the macula. The constant exposure of endothelial cells to pro-angiogenic factors, such as VEGFα, result in the formation of immature, semi-differentiated and fragile blood vessels which have a tendency to leak and bleed. Without wanting to be bound by theory, the present invention is based on the discovery that a compound of formula I as defined herein displays high selectivity towards the receptor tyrosine kinases (RTKs) PDGFRα, PDGFRβ and VEGFR2, the three main RTKs responsible for abnormal blood vessel growth in the context of MD. These receptor tyrosine kinases are high affinity cell surface receptors for polypeptide growth factors such as VEGFα. Accordingly, it is postulated that the compounds of the present invention may exhibit a wider therapeutic window than compounds or agents that do not distinguish between “diseased” and normal cells. This selectivity means the compounds of formula I, as well as pharmaceutically acceptable salt, solvate or prodrug thereof, may be particularly well suited for therapeutic application to patients with macular degeneration as they may be able to inhibit proliferation of only “diseased” cells; i.e. with high density of receptor tyrosine kinases. It is believed that the present compounds may be effective in blocking the sprouting of abnormal blood vessel formation, and accordingly be advantageous for treating MD and/or diabetic retinopathy.

The disease pathology of MD and/or diabetic retinopathy can be multi-factorial. In the treatment of MD and/or diabetic retinopathy, different therapies may be combined (i.e. combination therapies). The term “therapeutic agent”, “other therapeutic agent”, “another therapeutic agent”, “second therapeutic agent” and the like, as used herein is intended to include other therapeutic compounds or treatments which may be used in combination with the compound according to the present invention.

Thus, in accordance with the invention, compounds of formula I may be administered alone (i.e. as a monotherapy, such as a monotherapy of an angiogenesis-related disease or disorder). In alternative embodiments of the invention, however, compounds of formula I may be administered in combination with another therapeutic agent (e.g. another therapeutic agent for the treatment of an angiogenesis-related disease or disorder). In yet another embodiment of the invention, compounds of formula I may be administered as an adjuvant therapy after surgical treatment or as a neoadjuvant therapy before the main treatment (e.g. surgery) of the angiogenesis-related disorder or disease, either as a stand-alone compound or in combination with another therapeutic agent (e.g. another therapeutic agent for the treatment of an angiogenesis-related disease or disorder).

Second or other therapeutic agents useful in the treatment of eye disorders such as MD and/or diabetic retinopathy include, but are not limited to, angiogenesis inhibitors, vascular endothelial growth factor (VEGF) inhibitors, other receptor tyrosine kinase inhibitors, photodynamic therapy, laser photocoagulation, as well as other MD or AMD and/or diabetic retinopathy specific treatments. For example, a compound of formula I or a pharmaceutically acceptable salt, solvate or prodrug may be administered in combination with one or more VEGF inhibitors such as avastin, lucentis and/or macugen.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

For the avoidance of doubt, in cases in which the identity of two or more substituents in a compound of formula I may be the same, the actual identities of the respective substituents are not in any way interdependent.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, local ocular (i.e. subconjunctival, intravitreal, retrobulbar, intracameral), by any other parenteral route, in the form of a pharmaceutical preparation comprising the compound in a pharmaceutically acceptable dosage form. Particular modes of administration that may be mentioned include oral, topical, local ocular (i.e. subconjunctival, intravitreal, retrobulbar, intracameral), intravenous, cutaneous, subcutaneous, nasal, intramuscular or intraperitoneal administration.

When a compound of formula I is used to treat an eye disease or disorder, the compound of formula I is typically administered topically to the eye by or local ocular administration. Thus, in an embodiment, the compound of formula I or a pharmaceutically acceptable salt, solvate or prodrug is injected directly to the eye, and in particular the vitreous of the eye. The compound, composition or combination of the invention can be administered to the vitreous of the eye using any intravitreal or transscleral administration technique. For example, the compound, composition or combination can be administered to the vitreous of the eye by intravitreal injection. Intravitreal injection typically involves administering a compound of the invention or a pharmaceutically acceptable salt, solvate or prodrug in a total amount between 0.1 ng to 10 mg per dose.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringers solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants. By way of example, the compound, composition or combination can be dissolved in a pharmaceutically effective carrier and be injected into the vitreous of the eye with a fine gauge hollow bore needle (e.g., 30 gauge, ½ or ⅜ inch needle) using a temporal approach (e.g., about 3 to about 4 mm posterior to the limbus for human eye to avoid damaging the lens).

In an embodiment, a compound of formula I or a pharmaceutically acceptable salt, solvate or prodrug thereof may be formulated in a saline solution and injected into the vitreous of the eye.

Although intravitreal administration is a likely form of administration to the eye, the present invention also includes other modes of administration including topical or intravenous administration. For example, solutions or suspensions of the compound, composition or combinations of the invention may be formulated as eye drops, or as a membranous ocular patch, which is applied directly to the surface of the eye. Topical application typically involves administering the compound of the invention in an amount between 0.1 ng and 100 mg.

In another embodiment, the compound of formula (I) or a pharmaceutically acceptable salt, solvate or prodrug is provided on the surface of the eye. The compound may be provided on the surface of the eye as an eye drop, in particular as an eye drop composition or combination. The compound, composition or combinations of the invention can be administered to the surface of the eye using any known administration technique. For example, the compound or combinations can be administered to the surface of the eye by dripping the formulation onto the eye.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

Other compounds of formula I may be prepared in accordance with techniques that are well known to those skilled in the art, for example as described hereinafter in the examples section.

Compounds of the invention may be isolated from their reaction mixtures using conventional techniques (e.g. recrystallisation, column chromatography, preparative HPLC, etc.).

In the processes described hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described hereinafter may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

As used herein, the term “functional groups” means, in the case of unprotected functional groups, hydroxy-, thiolo-, amino function, carboxylic acid and, in the case of protected functional groups, lower alkoxy, N—, O—, S— acetyl, carboxylic acid ester.

Also disclosed herein are compounds of formula I where the linker group is of formula:

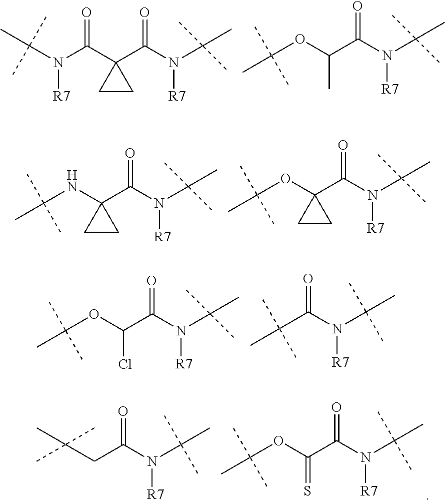

Compounds of formula I comprising these linker groups may be useful in the treatment of eye diseases and disorders in accordance with the invention. Such compounds may also be suitable for treating cancers such as prostate cancer, colon cancer, rectal cancer, colorectal cancer, acute myeloid leukaemia or chronic myelomonocytic leukaemia.

Compounds having linkers of the following formula may also be useful in the treatment of cancers such as prostate cancer, colon cancer, rectal cancer, colorectal cancer, acute myeloid leukaemia or chronic myelomonocytic leukaemia.

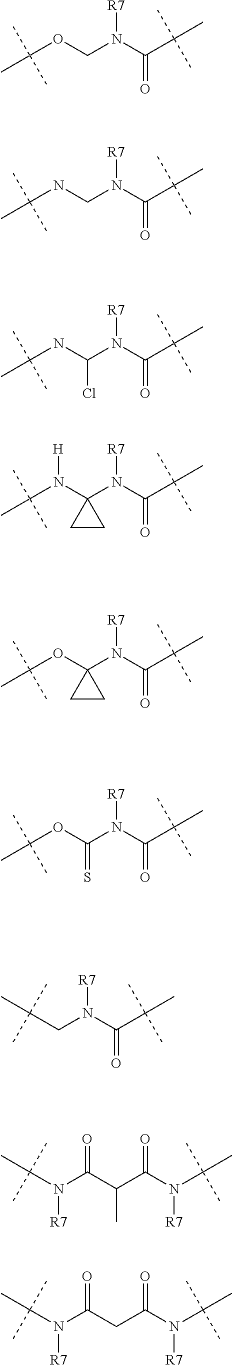

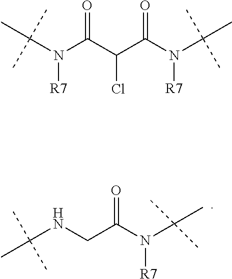

Second or other therapeutic agents useful in the treatment of cancers such as prostate cancer, colon cancer, rectal cancer, colorectal cancer, acute myeloid leukaemia or chronic myelomonocytic leukaemia include therapeutic agents useful in the treatment of hyperproliferative diseases or disorders, for example chemotherapy drugs. Examples of second or other therapeutic agents which can be used alongside a compound of formula I in the treatment of cancer include actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, Avastin (bevacizumab), bevacizumab, Camptosar (irinotecan hydrochloride), cetuximab, Cyramza (ramucirumab), Erbitux (cetuximab), 5-FU (fluorouracil injection), Fusilev (leucovorin calcium), Ipilimumab, irinotecan hydrochloride, Keytruda (pembrolizumab), leucovorin calcium, Lonsurf (trifluridine and tipiracil hydrochloride), Mvasi (bevacizumab), nivolumab, Opdivo (nivolumab), panitumumab, pembrolizumab, ramucirumab, regorafenib, Stivarga (regorafenib), trifluridine and tipiracil hydrochloride, Vectibix (panitumumab), Yervoy (ipilimumab), Zaltrap (ziv-aflibercept), abiraterone acetate, apalutamide, bicalutamide, cabazitaxel, Casodex (bicalutamide), degarelix, Eligard (leuprolide acetate), enzalutamide, Erleada (apalutamide), Firmagon (degarelix), flutamide, goserelin acetate, Jevtana (cabazitaxel), leuprolide acetate, Lupron (leuprolide acetate), Lupron Depot, (leuprolide acetate), mitoxantrone hydrochloride, Nilandron (nilutamide), Provenge (sipuleucel-T), radium 223 dichloride, Xofigo (radium 223 dichloride), Xtandi (enzalutamide), Zoladex (goserelin acetate), and Zytiga (abiraterone acetate).

The below examples illustrate the invention and are not to be construed as limitative.

### EXAMPLES

Compounds of the examples were prepared by the synthetic routes shown.

### Example 1

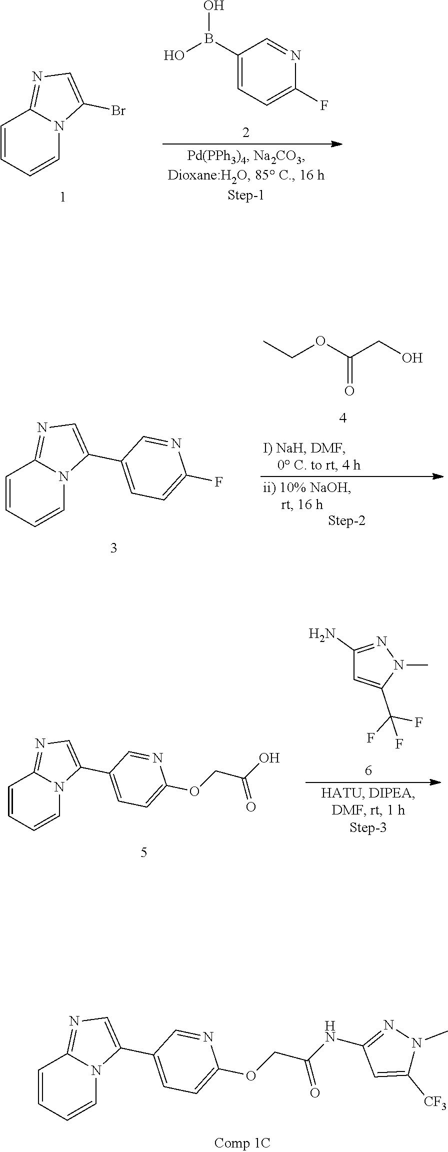

LCMS (ESI) m/z=417.05 [M+H]⁺; HPLC: RT 5.52, Purity: 95.17%; ¹H NMR (400 MHz, DMSO-d₆) δ=11.00 (s, 1H), 8.46 (d, J=6.9 Hz, 1H), 8.38 (d, J=2.0 Hz, 1H), 8.07 (dd, J=2.2, 8.6 Hz, 1H), 7.75 (s, 1H), 7.65 (d, J=9.4 Hz, 1H), 7.26-7.33 (m, 1H), 7.12 (d, J=8.4 Hz, 1H), 6.92-6.98 (m, 2H), 5.01 (s, 2H), 3.88 (s, 3H).

### Example 2

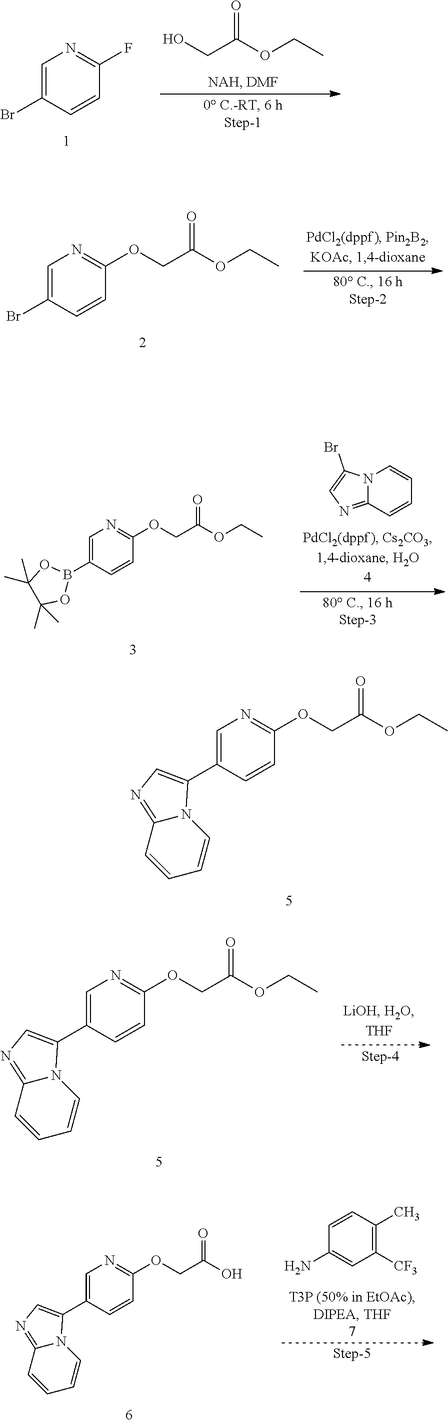

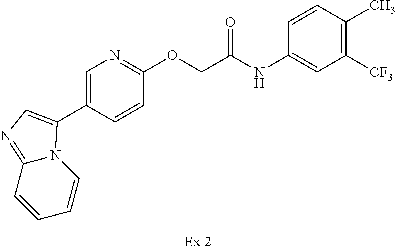 [SMILES: Cc1ccc(NC(=O)COc2ccc(-c3cnc4ccccn34)cn2)cc1C(F)(F)F]

### Example 3

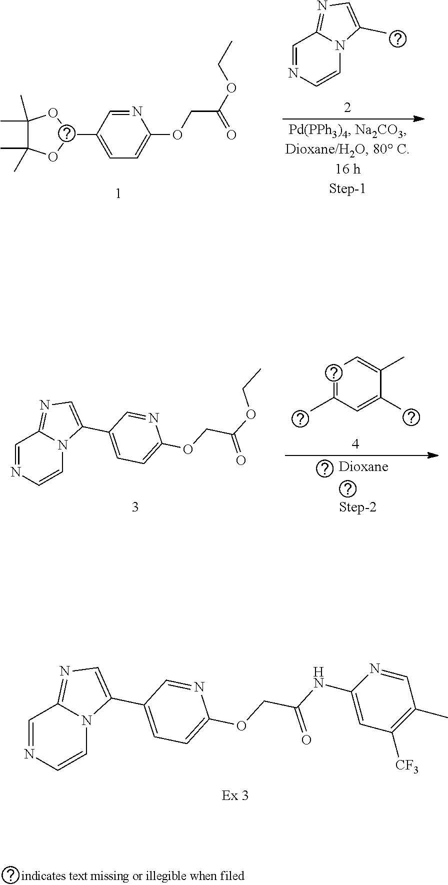

LCMS (ESI) m/z=429.00 [M+H]⁺; HPLC: RT 7.70, Purity: 99.29%; ¹H NMR (400 MHz, DMSO-d₆) δ=11.12 (br s, 1H), 9.14 (d, J=1.5 Hz, 1H), 8.56 (dd, J=1.2, 4.6 Hz, 1H), 8.45-8.49 (m, 2H), 8.33 (s, 1H), 8.15 (dd, J=2.4, 8.3 Hz, 1H), 8.05 (s, 1H), 7.91 (d, J=4.4 Hz, 1H), 7.16 (d, J=8.8 Hz, 1H), 5.11 (s, 2H), 2.38 (s, 3H).

### Example 4

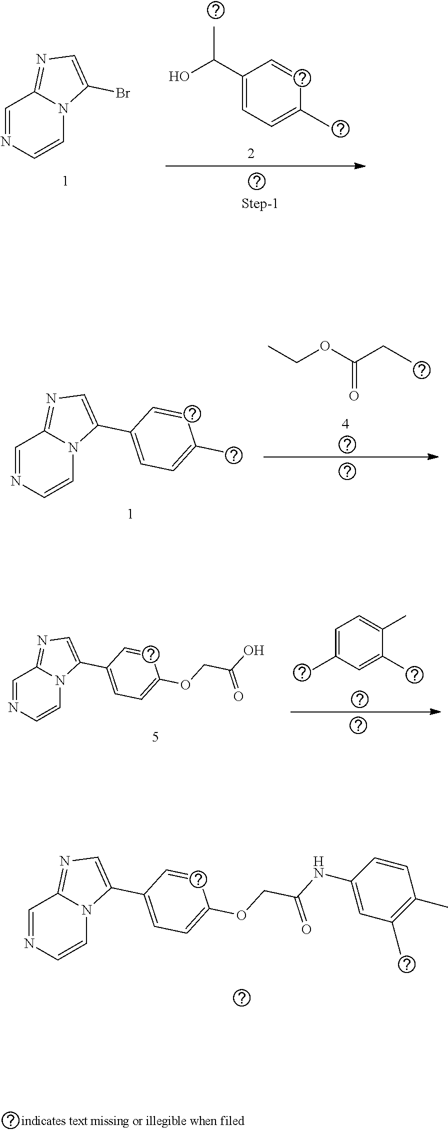

LCMS (ESI) m/z=428.20 [M+H]⁺; HPLC: RT 7.88, Purity: 98.07%; ¹H NMR (400 MHz, DMSO-d₆) δ=10.41 (s, 1H), 9.14 (s, 1H), 8.56 (d, J=4.9 Hz, 1H), 8.50 (d, J=2.0 Hz, 1H), 8.16 (dd, J=2.5, 8.4 Hz, 1H), 8.03-8.07 (m, 2H), 7.92 (d, J=4.4 Hz, 1H), 7.73 (d, J=8.4 Hz, 1H), 7.39 (d, J=8.4 Hz, 1H), 7.18 (d, J=8.9 Hz, 1H), 5.03 (s, 2H), 2.38 (s, 3H).

### Example 5

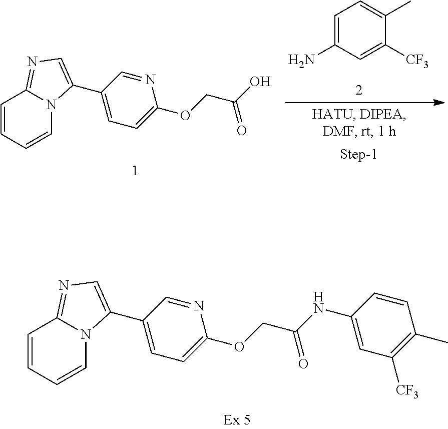 [SMILES: O=C(O)COc1ccc(-c2cnc3ccccn23)cn1 ; Cc1ccc(N)cc1C(F)(F)F ; Cc1ccc(NC(=O)COc2ccc(-c3cnc4ccccn34)cn2)cc1C(F)(F)F]

LCMS (ESI) m/z=427.30 [M+H]⁺; HPLC: RT 6.22, Purity: 99.78%; ¹H NMR (400 MHz, DMSO-d₆) δ=10.40 (s, 1H), 8.48 (d, J=6.9 Hz, 1H), 8.41 (d, J=2.0 Hz, 1H), 8.09 (dd, J=2.5, 8.4 Hz, 1H), 8.05 (d, J=2.0 Hz, 1H), 7.79 (s, 1H), 7.73 (d, J=8.4 Hz, 1H), 7.67 (d, J=8.9 Hz, 1H), 7.38 (d, J=8.4 Hz, 1H), 7.36-7.41 (m, 1H), 7.15 (d, J=8.9 Hz, 1H), 6.98 (t, J=6.9 Hz, 1H), 5.02 (s, 2H), 2.38 (s, 3H).

### Example 6

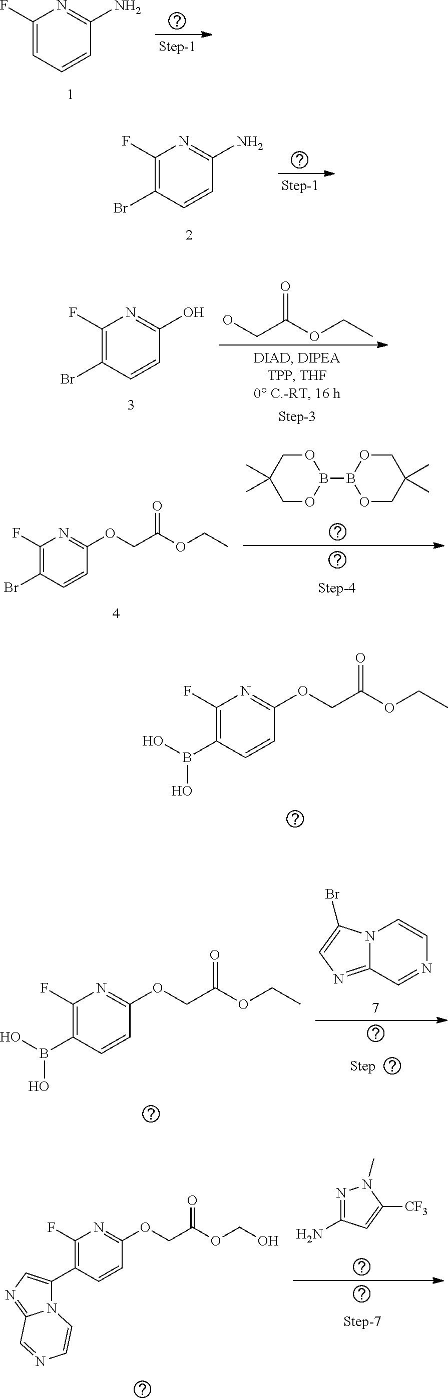

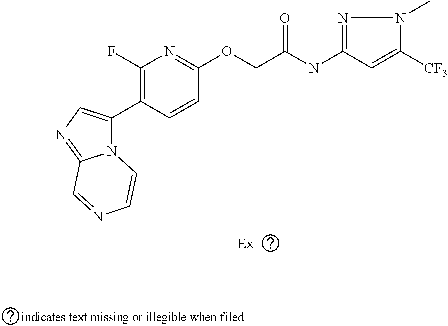 [SMILES: Cn1nc(NC(=O)COc2ccc(-c3cnc4cnccn34)c(F)n2)cc1C(F)(F)F]

### Example 7

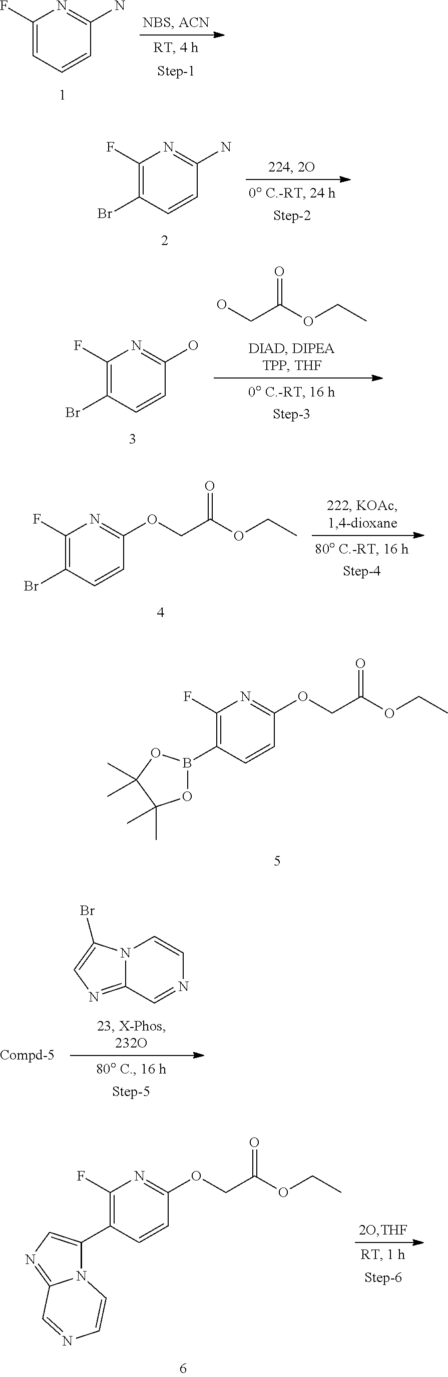

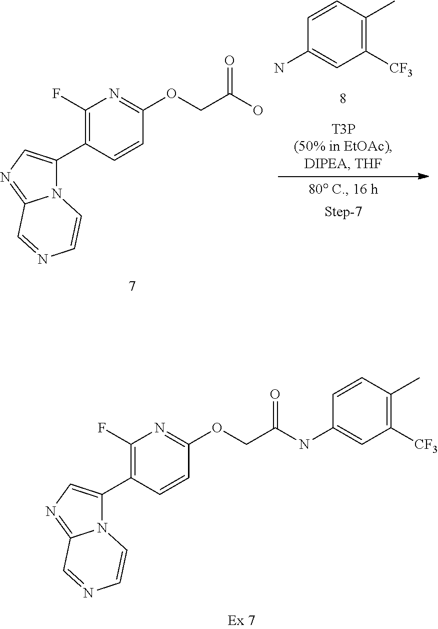 [SMILES: O=C(O)COc1ccc(-c2cnc3cnccn23)c(F)n1 ; Cc1ccc(N)cc1C(F)(F)F ; Cc1ccc(NC(=O)COc2ccc(-c3cnc4cnccn34)c(F)n2)cc1C(F)(F)F]

### Example 8

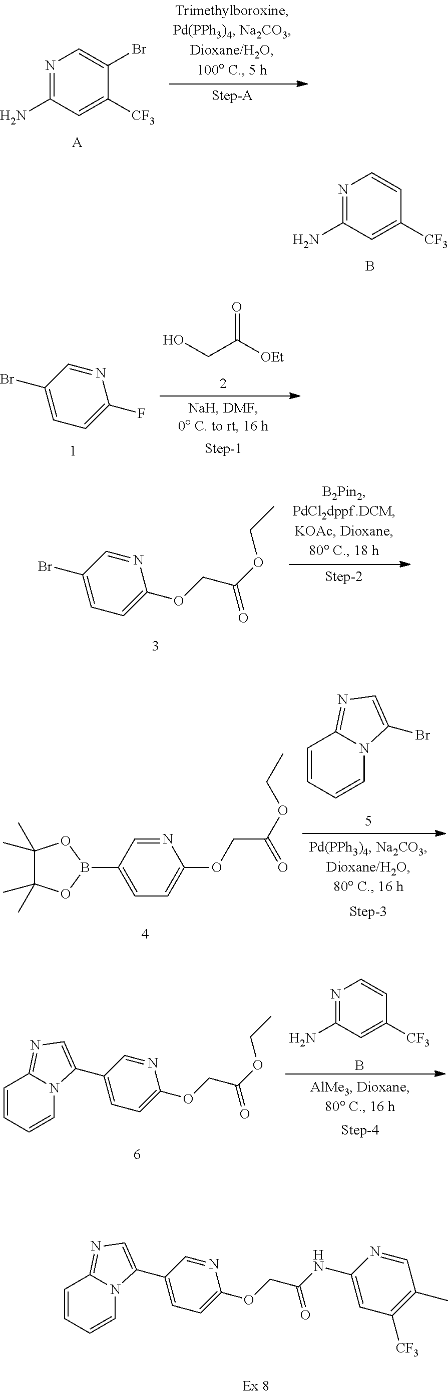

LCMS (ESI) m/z=428.20 [M+H]⁺; HPLC: RT 5.85, Purity: 99.51%; ¹H NMR (400 MHz, CHLOROFORM-d) δ=8.80 (br s, 1H), 8.57 (s, 1H), 8.38 (d, J=2.0 Hz, 1H), 8.25 (s, 1H), 8.20 (d, J=6.8 Hz, 1H), 7.86 (dd, J=2.2, 8.6 Hz, 1H), 7.65-7.77 (m, 2H), 7.12 (d, J=8.3 Hz, 1H), 6.86 (t, J=6.4 Hz, 1H), 5.07 (s, 2H), 2.44 (s, 3H).

### Example 9

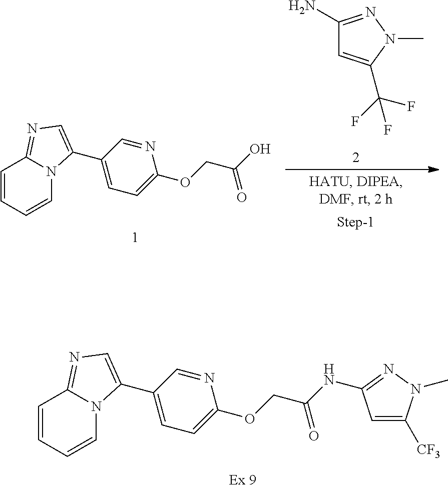 [SMILES: O=C(O)COc1ccc(-c2cnc3ccccn23)cn1 ; Cn1nc(N)cc1C(F)(F)F ; Cn1nc(NC(=O)COc2ccc(-c3cnc4ccccn34)cn2)cc1C(F)(F)F]

LCMS (ESI) m/z=418.20 [M+H]⁺; HPLC: RT 7.00, Purity: 98.45%; ¹H NMR (400 MHz, DMSO-d₆) δ=11.01 (s, 1H), 9.14 (s, 1H), 8.55 (d, J=4.9 Hz, 1H), 8.47 (d, J=2.5 Hz, 1H), 8.14 (dd, J=2.5, 8.9 Hz, 1H), 8.05 (s, 1H), 7.92 (d, J=4.9 Hz, 1H), 7.15 (d, J=8.4 Hz, 1H), 6.96 (s, 1H), 5.02 (s, 2H), 3.88 (s, 3H).

### Examples 10 to 19

Further compounds were produced by analogous methods and are listed in Table 1 below.

TABLE 1

| Example | Compound |
|---|---|
| 10 | 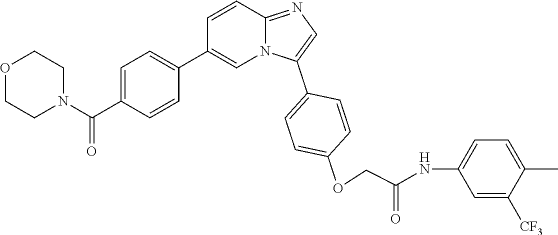 [SMILES: Cc1ccc(NC(=O)COc2ccc(-c3cnc4ccc(-c5ccc(C(=O)N6CCOCC6)cc5)cn34)cc2)cc1C(F)(F)F] |
| 11 | 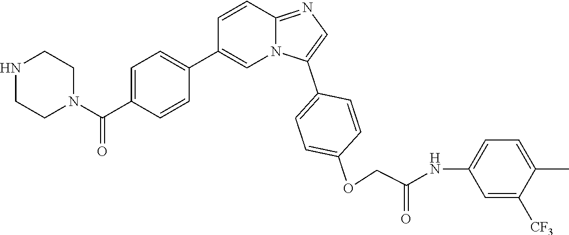 [SMILES: Cc1ccc(NC(=O)COc2ccc(-c3cnc4ccc(-c5ccc(C(=O)N6CCNCC6)cc5)cn34)cc2)cc1C(F)(F)F] |
| 12 | 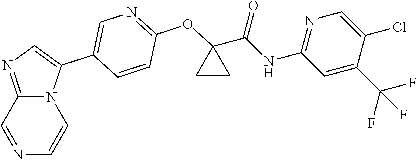 [SMILES: O=C(Nc1cc(C(F)(F)F)c(Cl)cn1)C1(Oc2ccc(-c3cnc4cnccn34)cn2)CC1] |
| 13 | 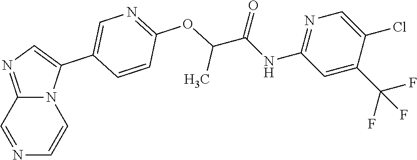 [SMILES: CC(Oc1ccc(-c2cnc3cnccn23)cn1)C(=O)Nc1cc(C(F)(F)F)c(Cl)cn1] |
| 14 | 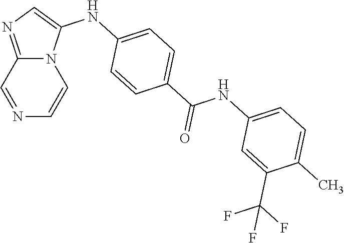 [SMILES: Cc1ccc(NC(=O)c2ccc(Nc3cnc4cnccn34)cc2)cc1C(F)(F)F] |
| 15 | 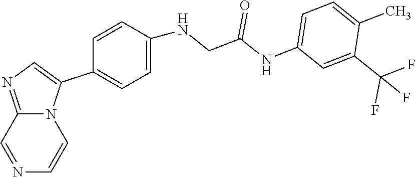 [SMILES: Cc1ccc(NC(=O)CNc2ccc(-c3cnc4cnccn34)cc2)cc1C(F)(F)F] |
| 16 | 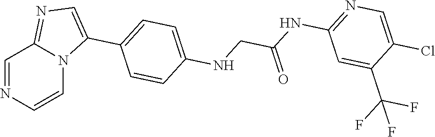 [SMILES: O=C(CNc1ccc(-c2cnc3cnccn23)cc1)Nc1cc(C(F)(F)F)c(Cl)cn1] |
| 17 | 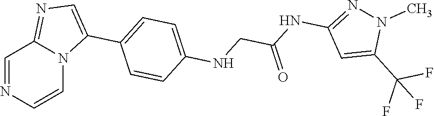 [SMILES: Cn1nc(NC(=O)CNc2ccc(-c3cnc4cnccn34)cc2)cc1C(F)(F)F] |
| 18 | 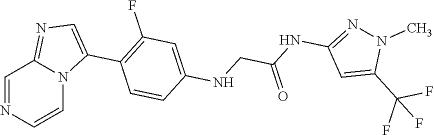 [SMILES: Cn1nc(NC(=O)CNc2ccc(-c3cnc4cnccn34)c(F)c2)cc1C(F)(F)F] |
| 19 | 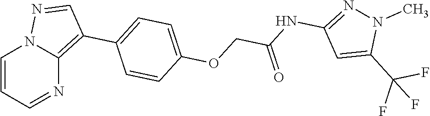 [SMILES: Cn1nc(NC(=O)COc2ccc(-c3cnn4cccnc34)cc2)cc1C(F)(F)F] |

### Example 12

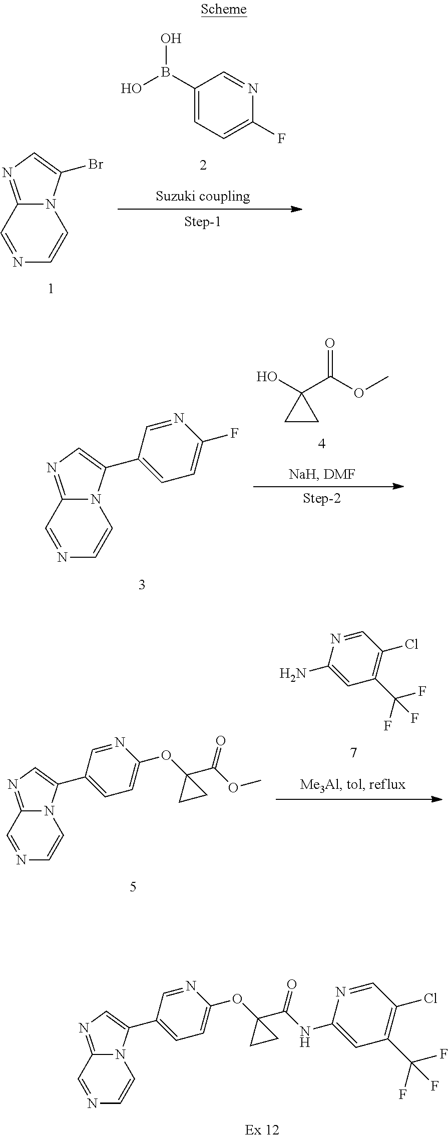

LCMS (ESI) m/z=475.00 [M+1]⁺; ¹H NMR (400 MHz, CDCl₃) δppm: 9.19 (s, 1H), 8.80 (s, 1H), 8.70 (s, 1H), 8.45 (d, 1H, J=2.8 Hz), 8.35 (s, 1H), 8.13 (d, 1H, J=4.4 Hz), 7.94 (d, 1H, J=4.4 Hz), 7.87-7.89 (m, 2H), 7.09 (d, 1H, J=8.4 Hz), 1.80-1.83 (m, 2H), 1.35-1.38 (m, 2H)

### Example 13

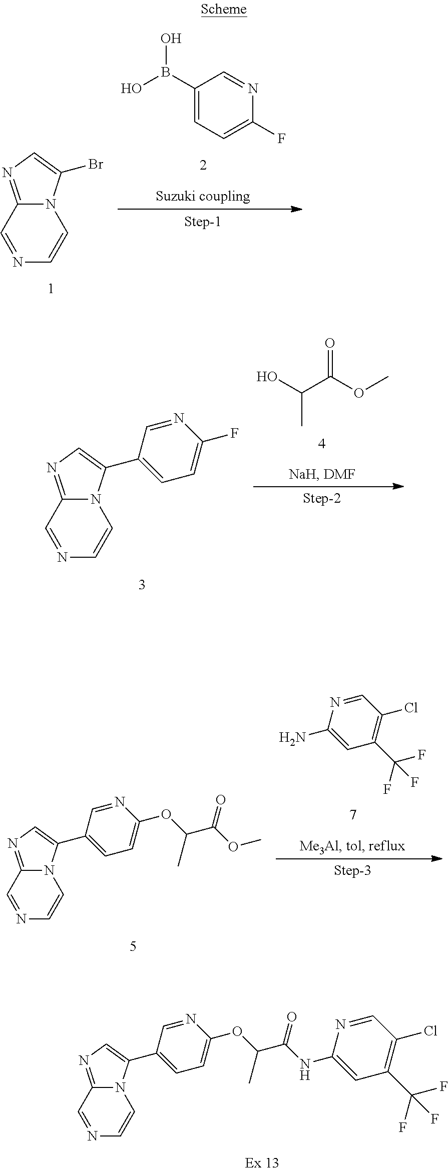

LCMS (ESI) m/z=463.2 [M+1]⁺; ¹H NMR (400 MHz, DMSO-d6) δppm: 11.46 (s, 1H), 9.12 (s, 1H), 8.72 (s, 1H), 8.53 (d, 1H, J=3.6 Hz), 8.50 (s, 1H), 8.43 (d, 1H, J=2.0 Hz), 8.14 (in, 1H), 8.03 (s, 1H), 7.90 (d, 1H, J=4.4 Hz), 7.13 (d, 1H, J=4.4 Hz), 5.53 (q, 1H, J=6.80 Hz), 1.60 (d, 1H, J=6.80 Hz).

### Example 14

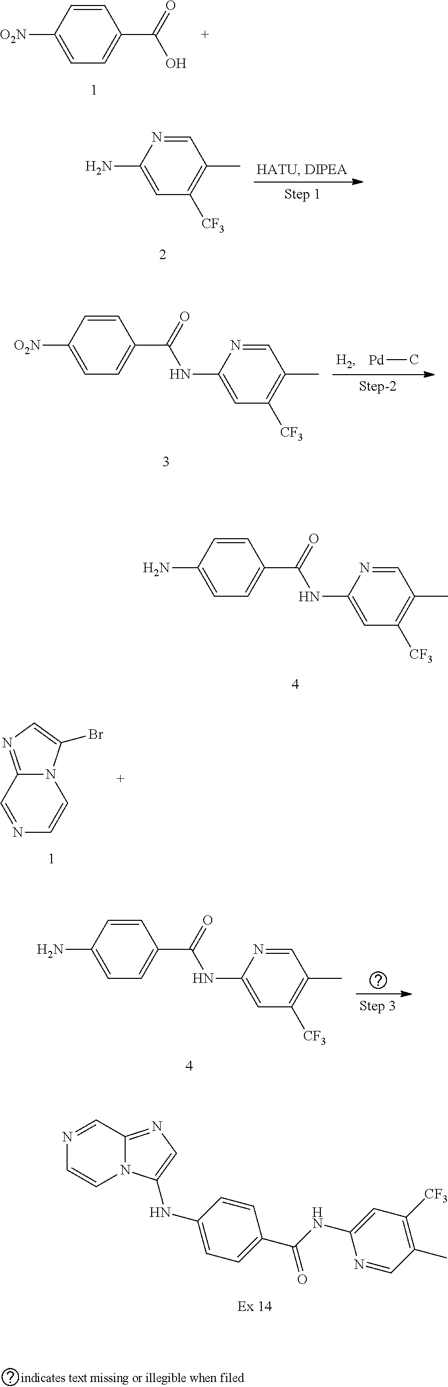

LCMS (ESI) m/z=412.05 [M+1]⁺; ¹H NMR (400 MHz, DMSO-d6) δppm: 10.17 (s, 1H), 9.06 (s, 1H), 8.81 (s, 1H), 8.16 (s, 1H), 8.08 (d, 1H, J=4.4 Hz), 7.80-7.95 (m, 4H), 7.81 (s, 1H), 7.38 (d, 1H, J=8.0 Hz), 6.75 (d, 1H, J=8.8 Hz), 2.39 (s, 3H)

### Example 15

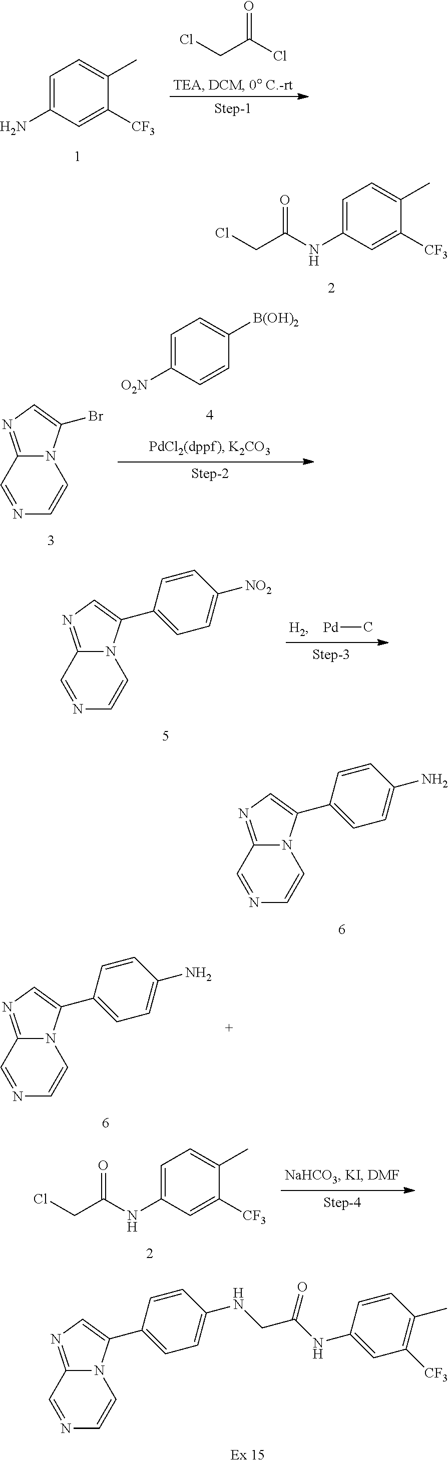

LCMS (ESI) m/z=426.05 [M+1]⁺; ¹H NMR (400 MHz, DMSO-d6) δppm: 10.31 (s, 1H), 9.06 (s, 1H), 8.48 (d, 1H, J=4.4 Hz), 8.05 (s, 1H), 7.85-7.88 (m, 2H), 7.75 (d, 1H, J=7.6 Hz), 7.46 (d, 1H, J=8.4 Hz), 7.36 (d, 1H, J=8.4 Hz), 6.78 (d, 1H, J=8.4 Hz), 6.49 (t, 1H, J=6.0 Hz), 3.97 (d, 1H, J=6.0 Hz), 2.37 (s, 3H)

### Example 16

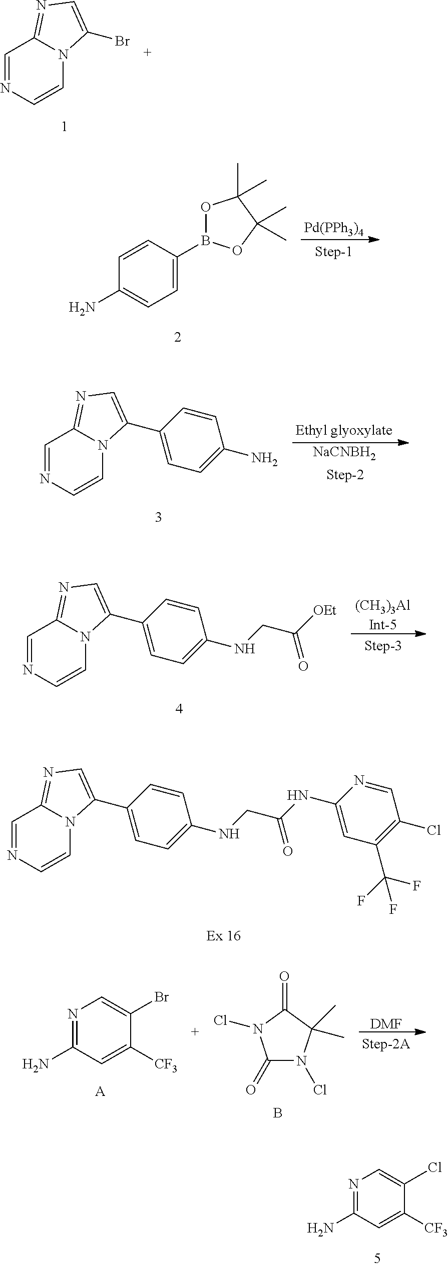

LCMS: 96.13%, m/z=448.2 [M+2H]⁺; ¹H NMR (DMSO-d₆ 400 MHz):δ 11.14-11.23 (m, 1H), 9.08 (d, J=1.34 Hz, JH), 8.72 (s, 1H), 8.54-8.59 (m, 1H), 8.50 (dd, J=1.41, 4.71 Hz, 2H), 7.85-7.91 (m, 2H), 7.47 (d, J=8.68 Hz, 2H), 6.80 (d, J=8.68 Hz, 2H), 6.51 (t, J=6.42 Hz, 1H), 4.12 (d, J=6.48 Hz, 2H);

### Example 17

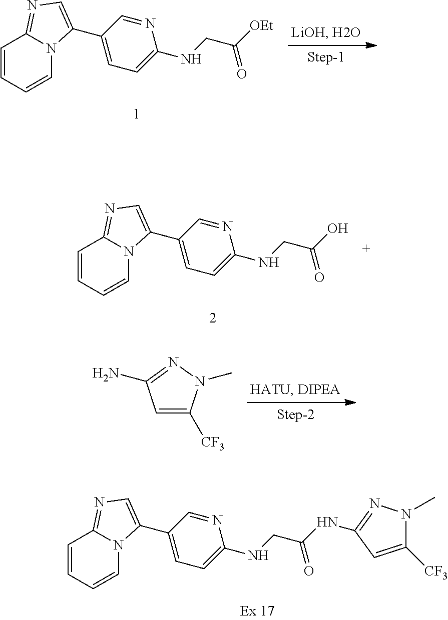 [SMILES: CCOC(=O)CNc1ccc(-c2cnc3ccccn23)cn1 ; O=C(O)CNc1ccc(-c2cnc3ccccn23)cn1 ; Cn1nc(N)cc1C(F)(F)F ; Cn1nc(NC(=O)CNc2ccc(-c3cnc4ccccn34)cn2)cc1C(F)(F)F]

LCMS: 98.08%, m/z=416.2 [M+H]⁺; ¹H NMR (DMSO-d₆, 400 MHz):δ 10.84 (s, 1H), 9.06 (d, J=1.34 Hz, 1H), 8.48 (dd, J=1.41, 4.71 Hz, 1H), 7.84-7.88 (m, 2H), 7.44 (d, J=8.56 Hz, 2H), 6.98 (s, 1H), 6.76 (d, J=8.56 Hz, 2H), 6.41-6.47 (m, 1H), 3.97 (d, J=6.36 Hz, 2H), 3.87 (s, 3H).

### Example 18

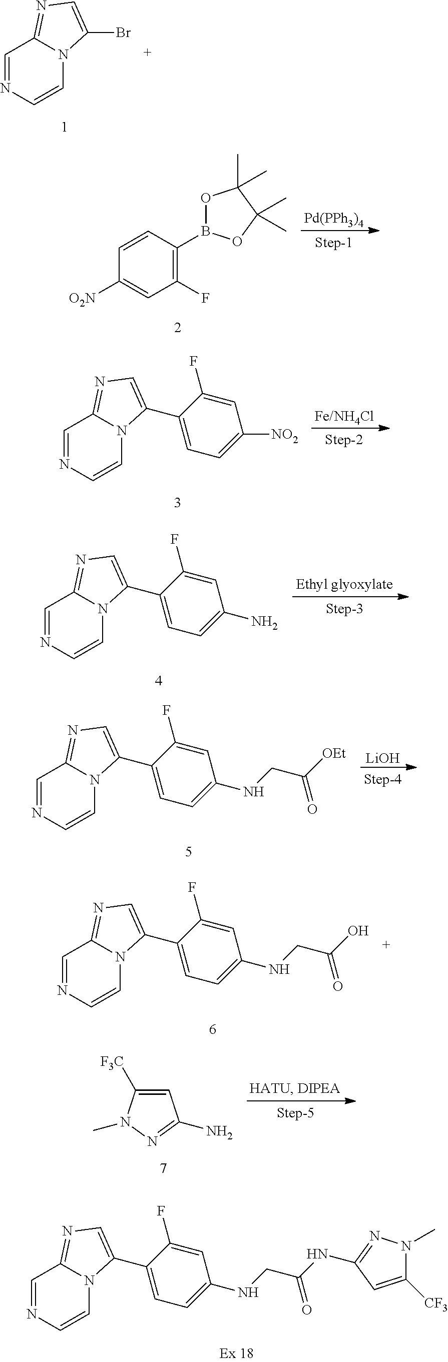

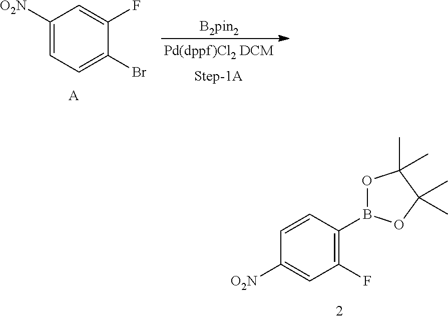

LCMS: 99.27%, m/z=434.2 [M+H]⁺; ¹H NMR (DMSO-d₆, 400 MHz):δ 10.90 (s, 1H), 9.09-9.11 (m, 1H), 8.19-8.22 (in, 1H), 7.85-7.90 (m, 2H), 7.33-7.39 (m, 1H), 6.99 (s, 1H), 6.76-6.81 (m, 1H), 6.57-6.63 (m, 2H), 4.00 (d, J=6.48 Hz, 2H), 3.88 (s, 3H);

Activity Data

Compounds of the Examples were tested in various assays as described below.

Determination of Activity in HEK293 Cell Line Model

Human Embryonic kidney cells (HEK293) were constructed to express human PDGFRWT under the control of constitutive CMV promoter. Cells were grown in the presence of 20 ng/ml PDGF (the ligand for the PDGFRβ) to activate the PDGFRβ. Different concentrations of the compounds of Examples 1 and 2 were added to study their effect on the PDGFRβ-mediated signaling. Autophosphorylation of PDGFRβ and Shp2 phosphorylation were monitored by Western blotting and were used as the markers for PDGFRβ signaling. β-Actin was used as an internal control.

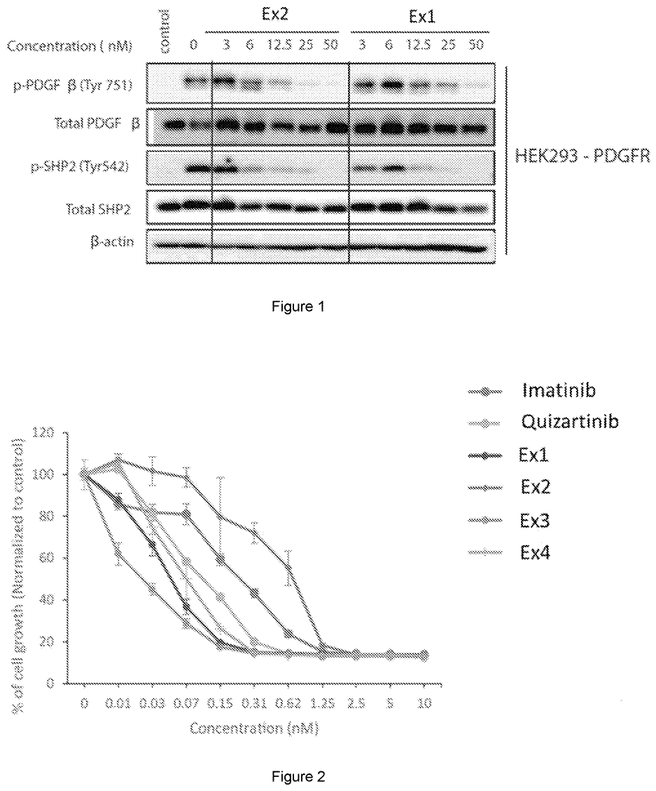

Results are shown in FIG. 1. It is clear that both Examples 1 and 2 are able to inhibit PDGFRβ signaling.

Cell Viability by MTS Assay

The effect of the compounds on cell viability were determined using MTS assay. MTS Cell Proliferation Assay Kit is a colorimetric method for sensitive quantification of viable cells in proliferation and cytotoxicity assay. The method is based on the reduction of MTS tetrazolium compound by viable cells to generate a colored formazan product that is soluble in cell culture media. BA/F3 cells expressing the receptor tyrosine kinase (either PDGFR [MTS Assay 1] or FIt3 [MTS Assay 2]) were cultured in standard culture conditions in a 96-well microtiter plate (final volume of 200 μl/well) in the absence or presence of the compound to be tested and were incubated for 20-48 hrs. MTS Reagent (20 μl/well) was added into each well & incubated for 0.5-4 hours at 37° C. in standard culture conditions. Absorbance at 490 nm was recorded using a plate reader.

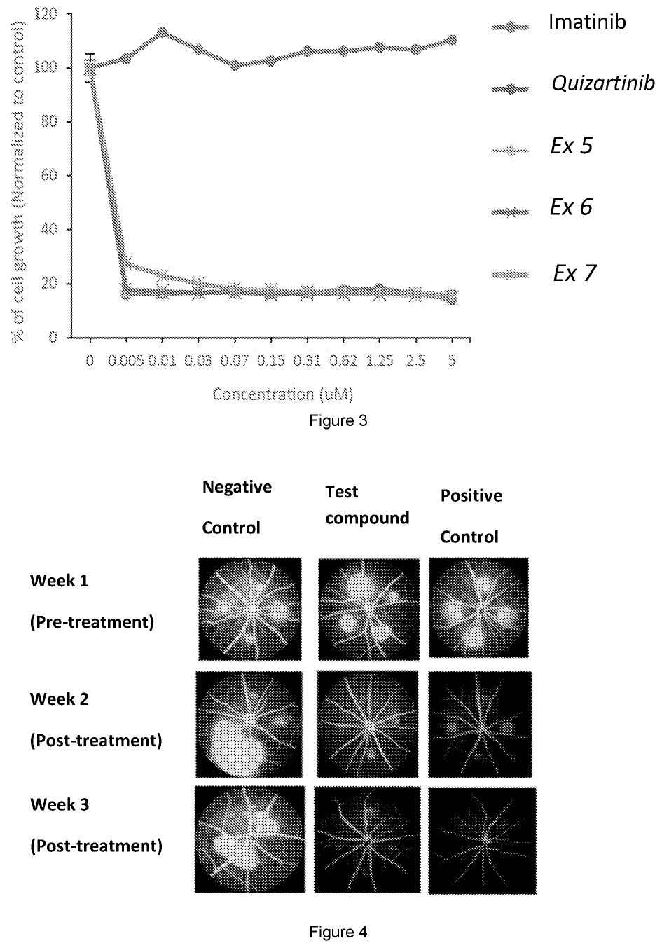

Results are shown in FIGS. 2 and 3 and Table 2 (where IC₅₀ indicates the concentration of compound required to reduce cell viability to 50%). It is clear that the compounds of Examples 1 to 7 have potent anti-tumour activity, with at least Examples 1, 3 and 4 having improved activity when compared to the imatinib and quizartinib controls.

In Vitro Activity and Selectivity Assay

To determine the inhibitory activity of compounds, in vitro kinase assays were performed on respective enzymes purified from Sf9 insect cells or E. coli as recombinant GST-fusion proteins or His-tagged proteins. The assay for all protein kinases contained 70 mM HEPES-NaOH pH 7.5, 3 mM MgCl₂, 3 mM MnCl₂, 3 μM Na-orthovanadate, 1.2 mM DTT, ATP (variable amounts, corresponding to the apparent ATP-Km of the respective kinase, [γ-33P]-ATP (approx. 9×1005 cpm per well), protein kinase and the peptide substrate. The IC₅₀ data for the tested compounds is provided in Table 2 below (a dash indicates that a compound was not tested).

L represents an IC₅₀ of less than 500 nM
M represents an IC₅₀ of between 500 nM and 5000 nM
H represents an IC₅₀ of more than 5000 nM

For the MTS Assay, the result indicates the concentration of compound required to reduce cell viability to 50%.

TABLE 2
IC₅₀ assay results

| | IC₅₀ (nM) | | | |
|---|---|---|---|---|
| Example | PDGFR-β | PDGFR-α | VEGFR-2 | MTS Assay 1 |
| 1 | L | L | L | L |
| 2/5 | — | — | — | L |
| 3/8 | L | L | L | L |
| 4 | — | — | — | L |
| 6 | — | — | — | M |
| 7 | — | — | — | L |
| 9 | — | — | — | L |
| 10 | — | — | — | L |
| 12 | H | H | H | — |
| 13 | M | M | M | — |
| 14 | H | H | H | — |
| 15 | L | L | L | — |
| 16 | L | L | L | — |
| 17 | L | L | L | — |
| 18 | L | L | L | — |
| 19 | L | L | L | — |

Effect on Capillary Formation In Vivo

FIG. 4 illustrates the efficacy of a compound of the present invention in Laser CNVmouse model for wet-AMD.

In vivo testing of a test compound according to the invention in CNV (Choroid Neo-Vascularization)-laser mouse model was performed. Eyes of young mice were treated with laser to create choroid damage which results in the leakage of blood vessels (shown as light areas of FIG. 4). In each mouse, one eye was injected with a predetermined amount of test compound while the other eye remained untreated to serve as ‘treatment (−)’ control. Mouse eyes were imaged intermittently for two weeks. The efficacy of the test compound is indicated by its ability to prevent the leakage of the blood vessels (reduction or disappearance of the light areas). The efficacy of the compound was very similar to the efficacy of the marketed product Eylea (Aflibercept).

### Reference Example 1: Additional Assay Results

TABLE 3
Additional IC₅₀ assay results

| | IC₅₀ (nM) | | | |
|---|---|---|---|---|
| Example | Flt3 | CDK8 | CDK19 | MTS Assay 2 |
| 1 | L | L | L | L |
| 2/5 | L | — | — | L |
| 3/8 | L | L | L | — |
| 4 | — | — | — | — |
| 6 | — | — | — | L |
| 7 | — | — | — | L |
| 9 | — | L | L | — |
| 10 | — | — | — | — |
| 12 | H | H | H | — |
| 13 | H | M | M | — |
| 14 | H | M | M | — |
| 15 | L | L | L | L |
| 16 | L | L | L | — |
| 17 | L | L | L | — |
| 18 | L | L | L | — |
| 19 | L | L | L | — |

In Table 3:
L represents an IC₅₀ of less than 500 nM
M represents an IC₅₀ of between 500 nM and 5000 nM
H represents an IC₅₀ of more than 5000 nM
For the MTS Assay, the result indicates the concentration of compound required to reduce cell viability to 50%.

1-20. (canceled)

21. A compound of formula 1: 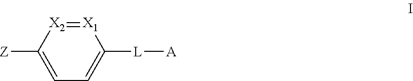
wherein:
X₁ and X₂ each independently represent N or CRₐ
Rₐ independently represents H, NH₂, halo, C₁₋₅ alkyl, C₁₋₅ alkoxy, C₂₋₅ alkenyl and C₂₋₅ alkynyl (which latter four groups are unsubstituted or substituted by one or more halo substituents);
A is selected from the group consisting of: 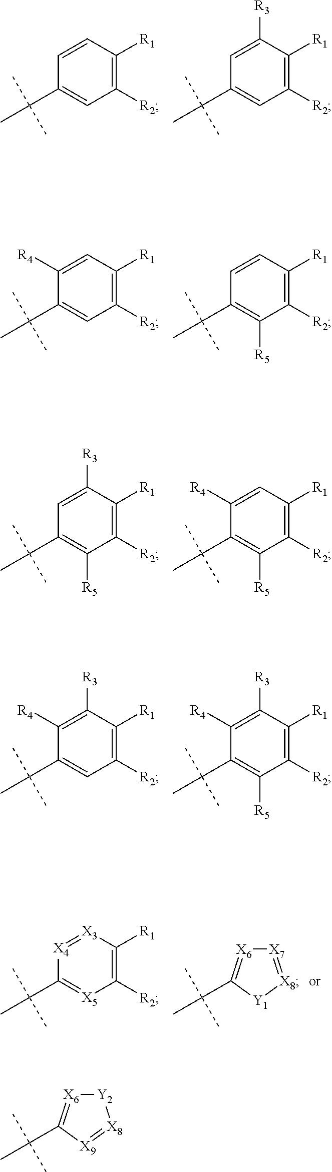
where:
the dotted line represents the point of attachment to the rest of the molecule;
each R₁ to R₅ is independently selected fro halo, C₁₋₅ alkyl, C₁₋₅ alkoxy, C₂₋₅ alkenyl, C₂₋₅ alkynyl, which latter four groups are unsubstituted or substituted by one or more halo substituents;
N represents N, CH or CR₃, where R₃ is as defined above, X₄ represents N, CH or CR₄, where R₄ is as defined above, X₅ represent N, CH or CR₅, where R₅ is defined above, provided that only one or two of X₃ to X₅ is N;
each X₆ to X₉ independently represents N, CH or CR₆, where each R₆ is independently selected from C₁₋₅ alkyl, C₁₋₅ alkoxy, C₂₋₅ alkenyl, C₂₋₅ alkynyl, which four groups are unsubstituted or substituted by one or more halo substituents;
wherein in any moiety A, one of R₁ to R₆ may be piperazine, methylpiperazine or ethylpiperazine, each of which may be connected to the rest of the moiety A via a carbon or nitrogen atom in the piperazine ring;
Y₁ represent NR_{N}, O or S;
Y₂ represents NR_{N}, NR_{Y} O or S;
R_{N} represents H, C₁₋₅ alkyl, C₂₋₅ alkenyl, C₂₋₅ alkynyl, which latter three groups are unsubstituted or substituted by one or more halo substituents;
R_{Y} represents piperazine, methylpiperazine or ethylpiperazine, each of which is connected to the nitrogen atom in Y₂ via a carbon atom in the piperazine ring;
L is a linking group of the formula:
-M-(CR_{L}R_{M})ₐ—C(O)—NR₇;
-M-(CR_{L}R_{M})ₐ—NR₇—C(O)—; or
-M-C(O)—(CR_{N}R_{O})—C(O)-M-
where M represents a covalent bond, O or NH;
R_{L} and R_{M} each independently represent H, methyl, ethyl, fluoro or chlore, or R_{L} and R_{M} together with the carbon atom to which they are attached, form a C₃ or C₄ cycloalkyl ring, carbonyl or thiocarbonyl group;
a represents O or 1;
R₇ and R_{7′} represent H or an optionally substituted alkyl group;
R_{N} and R_{O} each independently represent H, methyl, ethyl, fluoro or chlore;
Z represents a heterocycle selected from the group consisting of: 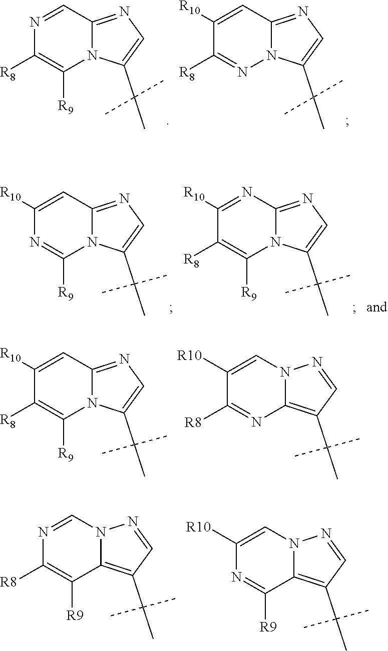
where:
the dotted line represents the point of attachment to the rest of the molecule, and Z is attached to the rest of the molecule via a covalent bond, or via a —O— or —NH— group;
each of R₈ to R₁₀ are independently selected from H, hydroxy, C₁ to C₅ alkyl, C₁ to C₅ alkoxy (which latter two groups are unsubstituted or substituted by one or more halo groups), OC(O)R₁₁, C(O)OR₁₂, C₂ to C₅ alkynyl (which is unsubstituted or substituted by one or more halo groups) or NR₁₃R₁₄, and O—(C₁₋₄ alkyleneyl)-O—C₁₋₄ alkyl,
and one of R₈ to R₁₀ may be a group of the formula: 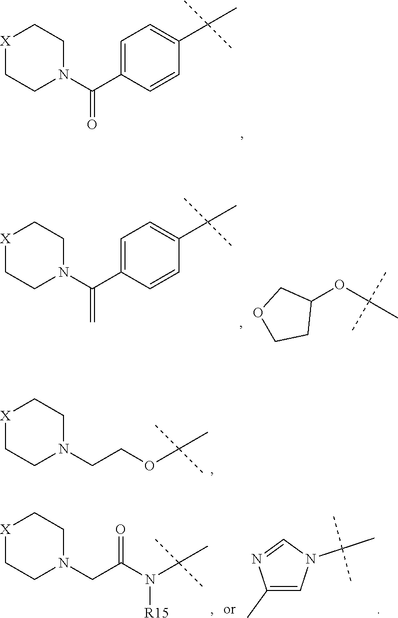
where X represents O, NR_{X},
R_{X} represents H or C₁₋₄ alkyl,
R₁₁ and R₁₂ each independently represent, at each occurrence, optionally substituted alkyl;
R₁₃ and R₁₄ each independently represent, at each occurrence, H or optionally substituted alkyl;
R₁₅ represents H or C₁₋₂ alkyl; or
a pharmaceutically acceptable salt, solvate or derivative thereof,

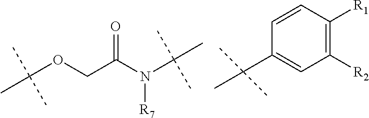

22. The compound according to claim 21, or a pharmaceutically acceptable salt, solvate or derivative thereof, wherein Rₐ independently represents H, NH₂, F, Cl, or C₁₋₃ alkyl, which C₁₋₃ alkyl group is unsubstituted or substituted by one, two or three fluoro or chlore substituents, optionally wherein Rₐ is H or F.

23. The compound according to claim 21, wherein X₁ is selected from N and CH, and X₂ is selected from CH and CF.

24. The compound according to claim 21, or a pharmaceutically acceptable salt, solvate or derivative thereof, wherein:
each R₁ to R₅ independently represents halo, C₁₋₃ alkyl, C₁₋₃ alkoxy, C₂₋₃ alkenyl and C₂₋₃ alkynyl (which four groups are unsubstituted or substituted by one or more halo substituents), optionally wherein each R₁ to R₅ independently represents fluoro, chlore, methyl or ethyl, which methyl and ethyl groups may be unsubstituted or substituted by one, two or three fluoro or chlore groups.

25. The compound according to claim 21, or a pharmaceutically acceptable salt, solvate or derivative thereof, wherein:
Y₁ and Y₂ independently represent O, NC₁₋₃ alkyl or NH; and/or
R₆ independently represents C₁₋₃ alkyl, C₁₋₃ alkoxy, C₂₋₃ alkenyl and C₂₋₃ alkynyl (which four groups are unsubstituted or substituted by one or more halo substituents), optionally wherein Y₁ and Y₂ independently represent O, NMe or NH, and/or
R₆ independently represents fluoro, chlore, methyl or ethyl, which methyl and ethyl groups may be unsubstituted or substituted by one, two or three fluoro or chlore groups.

26. The compound according to claim 24, or a pharmaceutically acceptable salt, solvate or derivative thereof, wherein each of R₁ to R₅ and R₆ independently represents methyl, trifluoromethyl, fluoro or chlore.

27. The compound according to claim 21, or a pharmaceutically acceptable salt, solvate or derivative thereof, wherein:
(a) each R₈ to R₁₀ independently represents H, hydroxy, Me, C₁₋₂ alkoxy (which is unsubstituted or substituted by one or more halo groups), OC(O)R₁₁, C(O)OR₁₂, C₂ to C₃ alkynyl (which is substituted by one or more halo groups), O—(C₁₋₂ alkyleneyl)-O—C₁₋₂ alkyl, or NR₁₃R₁₄,
R₁₁ and R₁₂ each independently represent methyl or ethyl,
R₁₃ and R₁₄ each independently represent H, methyl or ethyl; or
(b) one of R₈ to R₁₀ represents a group of the formula 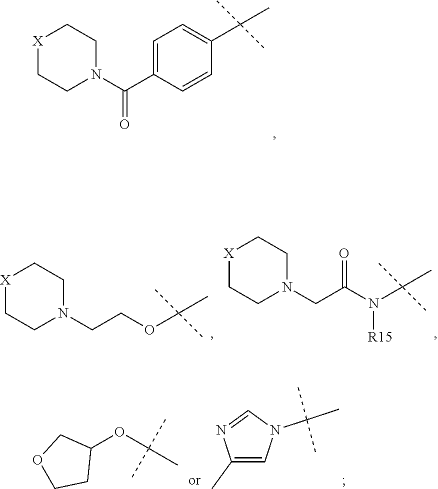
where X represents O, NH, or N—C₁₋₂ alkyl,
R₁₅ represents methyl,
and the remaining two of R₈ to R₁₀ are as defined in part (a).

28. The compound according to claim 21, or a pharmaceutically acceptable salt, solvate or derivative thereof, wherein Z represents a heterocycle selected from:

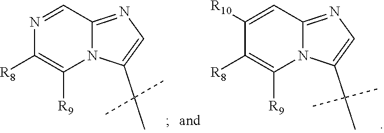

29. The compound according to claim 21, or a pharmaceutically acceptable salt, solvate or derivative thereof, wherein:
(a) when any of R₈ to R₁₀ is a C₁ to C₅ alkyl group, it is an unsubstituted methyl group; and/or
(b) when any of R₈ to R₁₀ is a C₂ to C₅ alkynyl group, it is a C₂ to C₅ alkynyl group which is substituted by one or more halo groups.

30. The compound according to claim 21, wherein:
R₉ and R₁₀, when present, are H, and

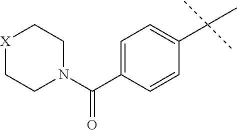

31. The compound according to claim 21, or a pharmaceutically acceptable salt, solvate or derivative thereof, wherein A is selected from the group consisting of: 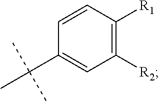 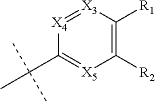
where only one of X₃ to X₅ is N; 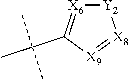
where only one of X₆, X₈ or X₉ is N; or 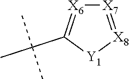
where only one of X₆, X₇ or X₈ is N.

32. The compound according to claim 31, wherein A is selected from the group consisting of: 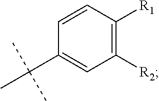 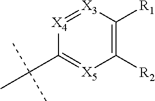
where only one of X₃ to X₅ is N; 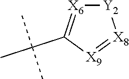
where only one of X₆, X₈ or X₉ is N;
and where when present:
R₁ is selected from Cl, CH₃ and H,
R₂ is CF₃,
X₃ and X₅ are CH,
X₄ is N,
X₆ is N,
X₈ and X₉ are CH,
Y₂ are selected from N—CH₃ and O.

33. The compound according to claim 21, or a pharmaceutically acceptable salt, solvate or derivative thereof, wherein:
M represents O or NH; and/or
R_{L} and R_{M} each independently represent H, methyl or chlore, or R_{L} and R_{M} together represent thiocarbonyl or cyclopropyl; and/or
a represents 1.

34. The compound according to claim 21, or a pharmaceutically acceptable salt, solvate or derivative thereof, wherein L represents: 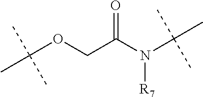
where the dotted lines represent the point of attachment to the rest of the molecule.

35. The compound according to claim 21 which is selected from: 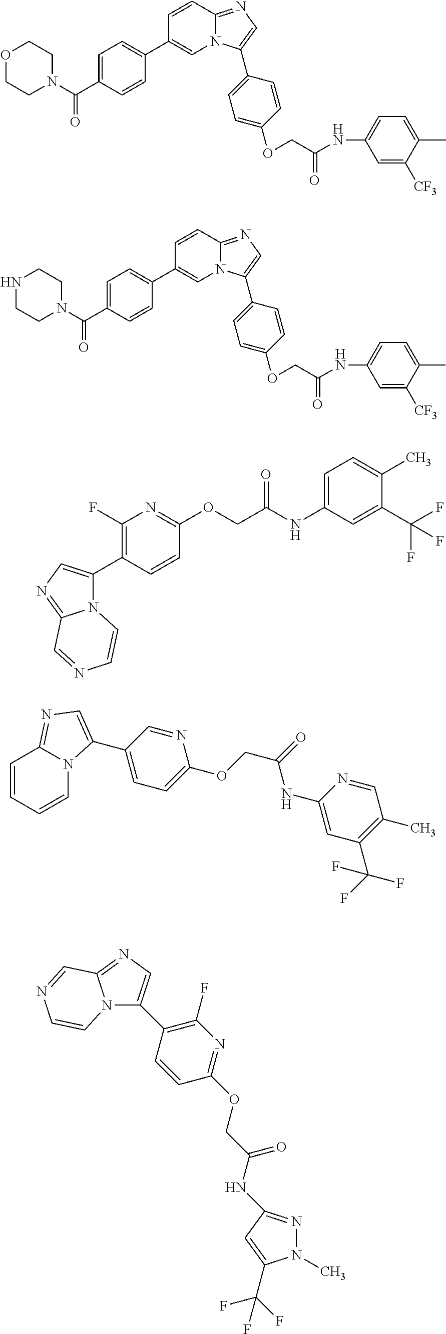 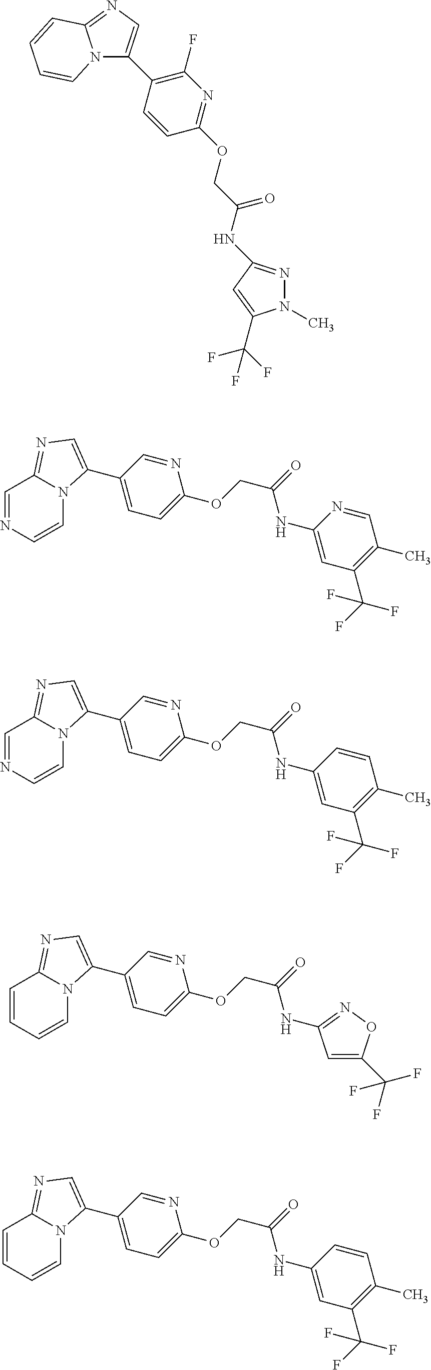 [SMILES: Cn1nc(NC(=O)COc2ccc(-c3cnc4ccccn34)c(F)n2)cc1C(F)(F)F ; Cc1cnc(NC(=O)COc2ccc(-c3cnc4cnccn34)cn2)cc1C(F)(F)F ; Cc1ccc(NC(=O)COc2ccc(-c3cnc4cnccn34)cn2)cc1C(F)(F)F ; O=C(COc1ccc(-c2cnc3ccccn23)cn1)Nc1cc(C(F)(F)F)on1 ; Cc1ccc(NC(=O)COc2ccc(-c3cnc4ccccn34)cn2)cc1C(F)(F)F] 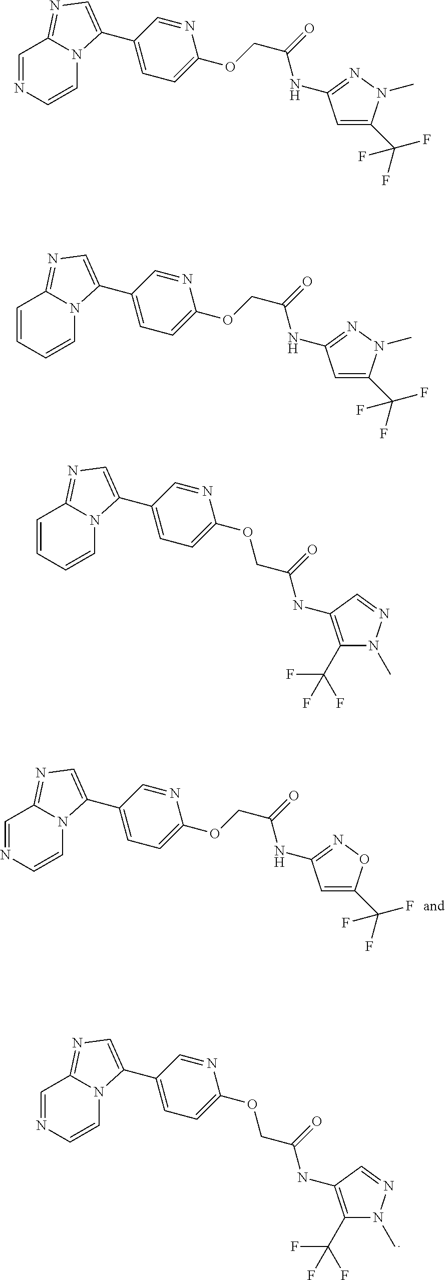 [SMILES: Cn1nc(NC(=O)COc2ccc(-c3cnc4cnccn34)cn2)cc1C(F)(F)F ; Cn1nc(NC(=O)COc2ccc(-c3cnc4ccccn34)cn2)cc1C(F)(F)F ; Cn1ncc(NC(=O)COc2ccc(-c3cnc4ccccn34)cn2)c1C(F)(F)F ; O=C(COc1ccc(-c2cnc3cnccn23)cn1)Nc1cc(C(F)(F)F)on1 ; Cn1ncc(NC(=O)COc2ccc(-c3cnc4cnccn34)cn2)c1C(F)(F)F] 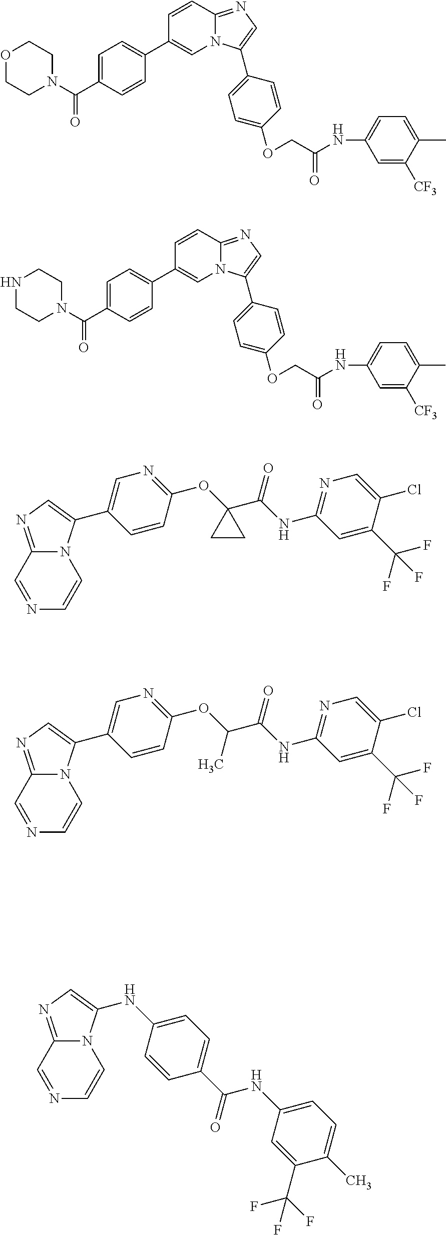 [SMILES: Cc1ccc(NC(=O)COc2ccc(-c3cnc4ccc(-c5ccc(C(=O)N6CCOCC6)cc5)cn34)cc2)cc1C(F)(F)F ; Cc1ccc(NC(=O)COc2ccc(-c3cnc4ccc(-c5ccc(C(=O)N6CCNCC6)cc5)cn34)cc2)cc1C(F)(F)F ; O=C(Nc1cc(C(F)(F)F)c(Cl)cn1)C1(Oc2ccc(-c3cnc4cnccn34)cn2)CC1 ; CC(Oc1ccc(-c2cnc3cnccn23)cn1)C(=O)Nc1cc(C(F)(F)F)c(Cl)cn1 ; Cc1ccc(NC(=O)c2ccc(Nc3cnc4cnccn34)cc2)cc1C(F)(F)F] 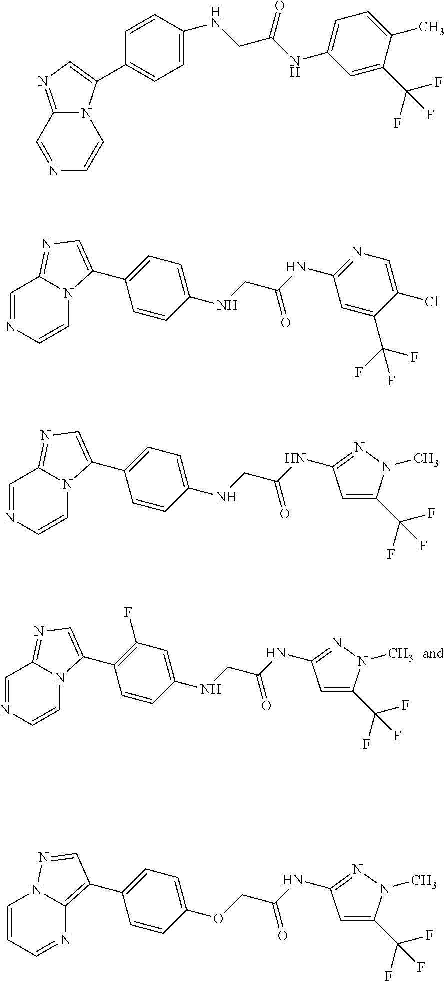 [SMILES: Cc1ccc(NC(=O)CNc2ccc(-c3cnc4cnccn34)cc2)cc1C(F)(F)F ; O=C(CNc1ccc(-c2cnc3cnccn23)cc1)Nc1cc(C(F)(F)F)c(Cl)cn1 ; Cn1nc(NC(=O)CNc2ccc(-c3cnc4cnccn34)cc2)cc1C(F)(F)F ; Cn1nc(NC(=O)CNc2ccc(-c3cnc4cnccn34)c(F)c2)cc1C(F)(F)F ; Cn1nc(NC(=O)COc2ccc(-c3cnn4cccnc34)cc2)cc1C(F)(F)F]
or a pharmaceutically acceptable salt, solvate or derivative thereof.

36. The compound according to claim 21, or a pharmaceutically acceptable salt, solvate or derivative thereof, wherein L is selected from: 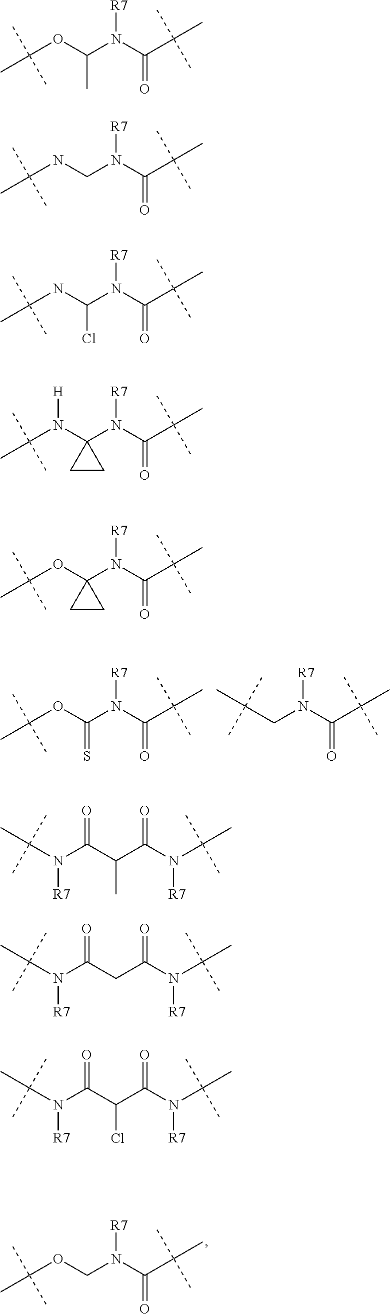
optionally wherein L is selected from

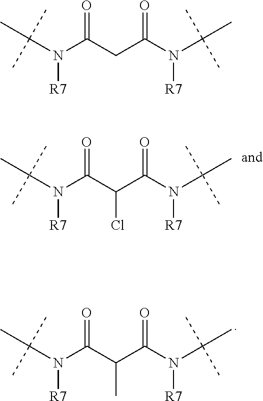

37. The compound according to claim 21, or a pharmaceutically acceptable salt, solvate or derivative thereof, wherein L is:

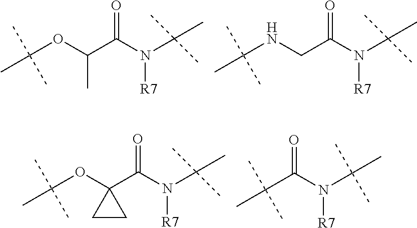

38. The compound according to claim 21, or a pharmaceutically acceptable salt, solvate or derivative thereof, wherein:
X₁ and X₂ each independently represent N or CRₐ
Rₐ independently represents H, NH₂, halo, C₁₋₅ alkyl, C₁₋₅ alkoxy, C₂₋₅ alkenyl and C₂₋₅ alkynyl (which latter four groups are unsubstituted or substituted by one or more halo substituents);
A is selected from the group consisting of: 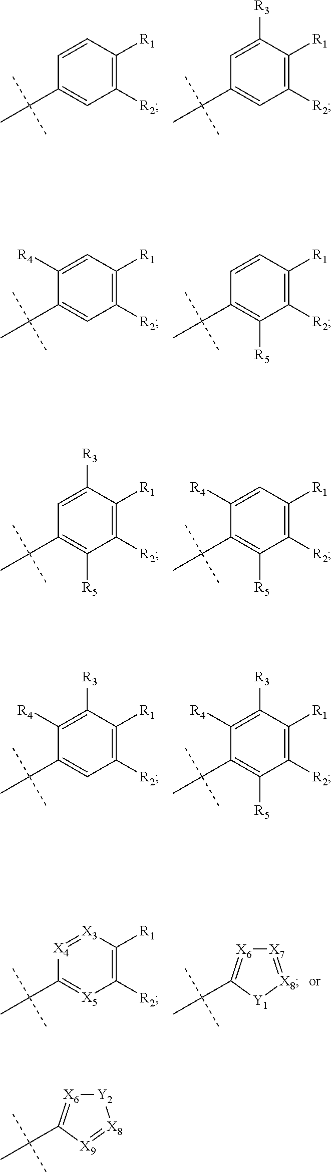
where:
the dotted line represents the point of attachment to the rest of the molecule;
each R₁ to R₅ is independently selected from halo, C₁₋₅ alkyl, C₁₋₅ alkoxy, C₂₋₅ alkenyl, C₂₋₅ alkynyl, which latter four groups are unsubstituted or substituted by one or more halo substituents;
X₃ represents N, CH or CR₃, where R₃ is as defined above, N represents N, CH or CR₄, where R₄ is as defined above, X₅ represents N, CH or CR₅, where R₅ is as defined above, provided that only one or two of X₃ to X₅ is N;
each X₆ to X₉ independently represents N, CH or CR₆, where each R₆ is independently selected from C₁₋₅ alkyl, C₁₋₅ alkoxy, C₂₋₅ alkenyl, C₂₋₅ alkynyl, which four groups are unsubstituted or substituted by one or more halo substituents;
Y₁ and Y₂ each independently represent NR_{N}, O or S;
R_{N} represents H, C₁₋₅ alkyl, C₂₋₅ alkenyl, C₂₋₅ alkynyl, which latter three groups are unsubstituted or substituted by one or more halo substituents;
L is a linking group of the formula:
-M-(CR_{L}R_{M})ₐ—C(O)—NR₇; or
-M-(CR_{L}R_{M})ₐ—NR_{7′}—C(O)—;
where M represents a covalent bond, O or NH;
R_{L} and R_{M} each independently represent H, methyl, ethyl, fluoro or chlore, or R_{L} and R_{M} together form a C₃ or C₄ cycloalkyl ring, carbonyl or thiocarbonyl group;
a represents O or 1;
R₇ and R_{7′} represent H or an optionally substituted alkyl group;
Z represents a heterocycle selected from the group consisting of: 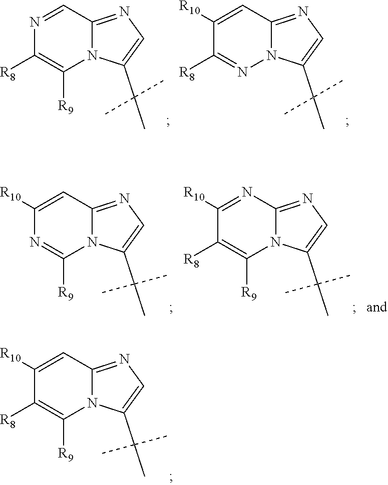
where:
the dotted line represents the point of attachment to the rest of the molecule, and Z is attached to the rest of the molecule via a covalent bond, or via a —O— or —NH— group;
each of R₈ to R₁₀ are independently selected from H, Me, C₁ to C₅ alkoxy which is unsubstituted or substituted by one or more halo groups, OC(O)R₁₁, C(O)OR₁₂, C₂ to C₅ alkynyl substituted by one or more halo groups or NR₁₃R₁₄, and one of R₈ to R₁₀ may be a group of the formula 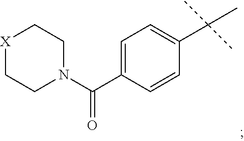
where X represents O or NH
R11 and R12 each independently represent, at each occurrence, optionally substituted alkyl;
R13 and R14 each independently represent, at each occurrence, H or optionally substituted alkyl;

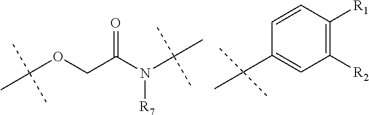

39. A method of treating one or more of macular degeneration, diabetic retinopathy, and angiogenesis, which method comprises administering a therapeutically effective amount of a compound of formula as defined in claim 21 or a pharmaceutically acceptable salt, solvate or derivative thereof.

40. A pharmaceutical composition comprising a compound of formula I as defined in claim 21 or a pharmaceutically acceptable salt, solvate or derivative thereof.
